(19) United States
(12) Patent Application Publication
(10) Pub. No.: US 2023/0001065 A1
(43) Pub. Date: Jan. 5, 2023

(54) IMPLANTABLE PUMP SYSTEM ENHANCEMENTS FOR USE IN CONDUCTNG DIRECT SODIUM REMOVAL THERAPY

(71) Applicant: Sequana Medical NV, Sint-Denijs Westrem (BE)

(72) Inventors: Andreas WIRTH, Simbach amInn (DE); Jeffrey TESTANI, Guilford, CT (US); Ian CROSBIE, London (GB); Oliver GOEDJE, Strasslach (DE)

(73) Assignee: Sequana Medical NV, Sint-Denijs Westrem (BE)

(21) Appl. No.: 17/930,064

(22) Filed: Sep. 6, 2022

Related U.S. Application Data
(63) Continuation-in-part of application No. 17/650,183, filed on Feb. 7, 2022, now Pat. No. 11,464,891.
Application No. 17/650,183 is a continuation-in-part of application No. 15/985,598, filed on May 21, 2018, now Pat. No. 10,918,778.
(60) Provisional application No. 62/510,652, filed on May 24, 2017.

(51) Int. Cl. A61M 1/34; A61M 1/28

(52) U.S. Cl. CPC A61M 1/3479; A61M 1/282; A61M 1/285; A61M 2205/3368; A61M 2205/502

(57) ABSTRACT

Enhanced systems and methods for performing Direct Sodium Removal (DSR) therapy are provided in which an implantable device includes a variable speed motor-driven pump that may be programmed to output different flow rates at different stages of a DSR therapy session, wherein the system monitors operational parameters of the pump and is configured to generate an alarm condition indicative of a fault that may be displayed on a patient's smartphone to permit corrective action, and in which a catheter set implanted with the implantable device enables a DSR solution may be instilled into the patient's peritoneal cavity using a peritoneal catheter that is subsequently used to remove the DSR solution and sodium-rich ultrafiltrate from the peritoneal cavity to the patient's bladder.

### CROSS-REFERENCE TO RELATED APPLICATIONS

This application is a continuation-in-part of U.S. patent application Ser. No. 17/650,183, filed Feb. 7, 2022, which is a continuation-in-part of U.S. patent application Ser. No. 17/174,855, filed Feb. 12, 2021, which is a continuation of U.S. patent application Ser. No. 15/985,598, filed May 21, 2018, now U.S. Pat. No. 10,918,778, which claims the benefit of priority of U.S. Provisional Application Ser. No. 62/510,652, filed May 24, 2017, the entire contents of which are incorporated herein by reference.

### FIELD OF THE INVENTION

The present invention relates generally to an enhanced implantable pump system for use in conducting direct sodium removal therapy in patients afflicted with heart failure or cardio-renal disease, and methods of use, in which a no or low sodium infusate is instilled into a patient's peritoneal cavity, and after a dwell time, the remaining infusate and accumulated ultrafiltrate and sodium is pumped to the patient's bladder using an implantable pump system.

### BACKGROUND

Patients afflicted with diverse forms of heart failure and/or cardio-renal disease are prone to the accumulation of additional sodium in body tissues and increased fluid retention. For example, in congestive heart failure, due to dysfunction of the left side or right side of the heart, or both, the body is unable to pump blood with normal efficiency, leading to the reduction of blood pressure in systemic circulation. In an attempt to increase blood pressure, the body retains sodium (and water), which leads to stasis or pooling of blood or fluid in the lungs or liver, edema and/or cardiac hypertrophy.

Methods, systems and compositions for directly removing excess sodium and water using a no or low sodium infusate instilled into a patient's peritoneal cavity are described in commonly-assigned U.S. Pat. No. 10,918,778, the entirety of which is hereby incorporated by reference. That patent describes a battery-powered pump, designed to be implanted subcutaneously, that includes an inlet catheter configured to be disposed within a patient's peritoneal cavity and an output catheter configured to be coupled to the patient's bladder. The pump is programmed to periodically actuate to move fluid from the peritoneal cavity to the bladder, where the fluid may be voided during urination.

As described in the above-incorporated patent, Direct Sodium Removal (“DSR”) Therapy may be conducted periodically, e.g., daily or once weekly, to remove excess sodium and fluid from a patient to reduce fluid-overload, edema, and to reduce cardiac effort in patients with heart failure, such as Heart Failure with reduced Ejection Fraction (“HFrEF”). To conduct such therapy, a quantity, e.g., up to 2 liters, of a no or low sodium DSR solution is instilled into the patient's peritoneal cavity for a dwell period, which may vary from a couple of hours up to 24 hours, depending on type of DSR infusate used. During the dwell period, the DSR solution creates a sodium gradient that draws excess sodium from the patient's tissues and/or bloodstream into the peritoneal cavity, along with a corresponding volume of ultrafiltrate, e.g., water. At the conclusion of the dwell period, the implantable pump is actuated to move the now sodium-rich DSR solution and ultrafiltrate from the peritoneal cavity to the bladder. As observed in the above-incorporated patent and in initial human clinical testing, DSR therapy can reduce physiologically significant amounts of sodium and fluid from the patient, thereby reducing fluid overload and improving cardiac function.

One aspect of DSR therapy, as described in the above patent, is that the dwell period for the DSR solution in the patient's peritoneal cavity may be based on the physician's prior clinical experience and assessment of the specific patient's physiology. In the above incorporated, commonly assigned, U.S. patent application Ser. No. 17/650,183, in one aspect of that invention an analyte sensor is described as associated with the implantable pump system for monitoring an analyte concentration, such as sodium ions, in the fluid in the patient's peritoneal cavity, and for automatically adjusting the dwell time prior to initiating fluid transfer to the patient's bladder to adapt the general DSR therapy to the physiologic needs of specific patients. For example, some patients may have a high level of excess sodium stored in extravascular spaces, e.g., in interstitial spaces and tissues, whereas others may not. Consequently, the above-incorporated application discloses adjusting a dwell period for a DSR solution in accordance with a monitored analyte concentration, or to transfer a specified amount of analyte from the peritoneal cavity to the bladder for a particular therapy session.

One feature of the implantable pump of the above-incorporated application is that the pump generally is operated at a substantially constant motor speed during actuation. In may occur, however, depending upon the specific patient's physiology and the type of DSR solution used, that fluid or sodium accumulation in the patient's peritoneal cavity may progress faster than anticipated by the physician. In such a case, it may be desirable to provide an implantable system with a variable motor speed, which may be adjusted manually or on a pre-programmed basis to pump fluid from the patient's peritoneal cavity at different flow rates during or subsequent to the DSR therapy session. For example, it may be desirable to accelerate fluid transfer to substantially empty the bulk of the instilled fluid and ultrafiltrate promptly after expiration of a nominal dwell time (or as indicated responsive to monitored analyte concentration) and then employ a lower pump speed to transfer subsequently accumulated fluid.

In connection with enabling variable pumping speeds, as mentioned above, it further would be desirable to provide a more robust alarm system for the implantable pump system. For example, whereas the implantable pump described in the above-identified patent employed a single flow rate, with the additional of multiple flow rates as discussed in the preceding paragraph, it would be desirable to monitor the pump speed to ensure that a desired pump speed is attained during a specified interval. If the desired pump speed is not attained, for example, due to proteinaceous buildup within the pump, it would be desirable for the processor controlling the implantable pump to generate an alarm that is communicated to the patient, e.g., via the external charging system or by communication with software loaded onto a patient's smartphone. The patient might then be presented with various options for rectifying the alarm condition.

In addition, the implantable pump system described in the above-identified patent included pressure sensors for monitoring intraabdominal pressure, and activating the implantable pump when the pressure exceeds a specified threshold value. In the context of DSR therapy, however, in view of the compliance of the abdominal cavity, it instead may be desirable to monitor and trigger pump actuation based on the time-dependent evolution of pressure within the peritoneal cavity. In this manner, the implantable pump could differentiate between pressure fluctuations caused by the patient coughing or bending over from pressure increases resulting from filling of the peritoneal cavity.

It also may be desirable to employ the catheter used for instilling the DSR fluid into the patient's peritoneal cavity during initiation of the DSR therapy to flush potential blockages from the inlet of the implantable pump.

Accordingly, it would be desirable to develop provide an implantable pump system for use in conducting DSR therapy sessions in which the implantable pump system provides additional flexibility in ease of use as well as enhanced monitoring and safety features.

### SUMMARY OF THE INVENTION

The present invention is directed to an implantable pump system having enhanced features and programming adapted for conducting DSR therapy for patients suffering from fluid and/or sodium overload, e.g., heart failure patients and patients afflicted with cardio-renal disease. In accordance with one aspect of the invention, an implantable pump system as described in commonly assigned U.S. Pat. No. 10,918,778 and/or U.S. patent application Ser. No. 17/650,183 is provided, which in addition provides additional enhancements for use in conducting DSR therapy, including variable programmable pump speeds, a set of more comprehensive set of alarm conditions and trouble-shooting options for resolving such alarm conditions, and increases ease of use for instilling a DSR solution during initiation of the DSR therapy.

In accordance with the principles of the present invention, an implantable pump system is provided that includes a programmable variable-speed implantable pump coupled to an inlet catheter having an inlet end configured to be disposed in a patient's peritoneal cavity and an outlet catheter having an outlet end configured to be disposed in the patient's bladder. The implantable pump is configured for wireless communication with an external charging and communication system. The external charging and communication system in turn may be configured to communicate with a software application installed on a physician monitoring and control system and optionally, a software application installed on the patient's smartphone, personal computer or tablet. In one preferred embodiment, such as described in the above-incorporated application, the implantable pump system includes sensor for measuring a concentration of an analyte, such as sodium, for detecting a concentration of an infusate component, such as dextrose or icodextrin, or an osmotic pressure or gradient, or for monitoring an amount of sodium removed from the peritoneal cavity to the bladder during a pumping session.

Further in accordance with the principles of the invention, the implantable pump includes a positive-displacement gear pump, such that the volume of fluid transferred during a pumping session may be accurately computed based on the number of gear rotations during a specified interval. According to one aspect of the invention, the implantable pump system includes an on-board processor that may be programmed by the patient's physician to operate the gear pump at different speeds. For example, the pump may operate at a first speed to rapidly transfer fluid from a patient's peritoneal cavity to the patient's bladder following completion of a predetermined dwell time, or in response to detection by the analyte sensor of an analyte level in the peritoneal cavity above a predetermined threshold. The implantable pump also may be programmed to operate thereafter at a substantially lower speed to periodically remove further fluid accumulations from the peritoneal cavity after the conclusion of the DSR therapy session. Further, the implantable pump may be operated at a selected speed specifically in response to a command transmitted by the patient, his or her caretaker or physician.

In accordance with another aspect of the invention, the implantable pump system includes an enhanced alarm monitoring and notification capability. In particular, to address the increased pump speed programming complexity, the implantable pump system preferably includes additional alarm monitoring, reporting and resolution capability. For example, a preferred embodiment may include programming for monitoring pump operation that promptly generates an alarm to notify the patient, via the external charging and control system or a smartphone application, that the pump output is below a specified threshold for output for a particular phase of the DSR session. In this case, the patient may be contact his or her physician, who may send commands via the smartphone application to the implantable pump system to increase pump speed for the remainder of the pumping session. Alternatively, if the alarm identifies a blockage in the gear pump due to the presence of particulate matter, the programming may notify the patient to request that the physician remotely activate a boost mode or jog mode to overcome and/or grind and remove the obstruction, as described below. Alternatively, the smartphone application may send such notifications directly to the patient's physician, as well as the patient. In a still further alternative embodiment, the alarm software of the implantable pump system may monitor intraabdominal pressure during instillation of the DSR fluid or during the dwell period, and notify the patient to request cessation of addition of DSR fluid to the peritoneal cavity or to prompt transfer of accumulated fluid to the patient's bladder.

In yet another aspect of the invention, the implantable pump system may include advanced algorithms for determining completion of a dwell period and when to activate the pump to transfer fluid from the peritoneal cavity to the bladder. The above-incorporated patent and application describe that the dwell period for retaining the DSR solution in the peritoneal cavity may be based on a specified time, e.g., 2 hours, attaining a threshold intraabdominal pressure, or attaining an analyte concentration level in the fluid in peritoneal cavity. In accordance with the present invention, actuation of the implantable pump further may be triggered by monitoring the rate of change of pressure within the peritoneal cavity over a specified time interval. In this manner, the pump will not be inadvertently triggered by transient increases in intraabdominal pressure, e.g., due to patient coughing or transient postural changes.

Further in accordance with the principles of the invention, the implantable pump system may include an improved fluid transfer set for flushing the peritoneal catheter when instilling DSR fluid into the patient's peritoneal cavity during initiation of the DSR session. In one preferred embodiment, the inlet catheter to the pump includes a tee-shaped junction, wherein a first side of the junction has an instillation line configured to be coupled to a subcutaneous port having a self-healing membrane, a second side of the junction couples to a peritoneal catheter configured to be positioned in the peritoneal cavity and a third port of the junction is configured to be coupled via tubing to the implantable pump inlet port. A valve optionally may be incorporated into the tee-shaped junction, which closes to prevent instilled fluid from flowing into the implantable pump during infusion of DSR solution, and which closes the instillation during pumping of fluid from the peritoneal cavity to the bladder.

In this manner, DSR solution, typically 0.5 up to 2 liters, may be instilled via a needle inserted into the subcutaneous port, so that it flows through the instillation line, the tee junction with optional valve and the peritoneal catheter into the peritoneal cavity, thereby flushing the peritoneal catheter of any debris. After instillation of the DSR solution is completed, the needle is withdrawn. Upon completion of the dwell period, as may be determined by expiration of a specified time, analyte concentration, or rate of change of intraabdominal pressure, the implantable pump is actuated so that fluid is withdrawn from the peritoneal cavity via the peritoneal catheter, the tee junction and the inlet catheter to the implantable pump. No fluid is drawn through the instillation line as the self-healing membrane on the subcutaneous port and valve, if present, will seal the instillation line. In this manner, the peritoneal catheter may be used to rapidly deliver DSR fluid into the peritoneal cavity while ensuring that the catheter remains free of blockage.

In an alternative embodiment, the improved fluid transfer set for instilling DSR fluid into the patient's peritoneal cavity during initiation of the DSR session may include a Y-shaped connector that may be assembled with the peritoneal catheter during implantation of the implantable device.

Other features of the inventive system and methods will be apparent with reference to the following description and figures.

### BRIEF DESCRIPTION OF THE DRAWINGS

FIG. 1 is a perspective view of an exemplary system constructed in accordance with the principles of the present invention for use in performing DSR therapy.

FIG. 2 is a schematic diagram of the electronic components of an exemplary embodiment of the implantable device.

FIGS. 3A and 3B are, respectively, a perspective view of the implantable device with the housing shown in outline and a perspective view of the obverse side of the implantable device with the housing and low water permeable filler removed.

FIGS. 4A, 4B, 4C and 4D are, respectively, an exploded perspective view of the drive assembly of the implantable device; front and plan views of the upper housing; and a perspective view of the manifold of an exemplary embodiment of the implantable device.

FIGS. 5A and 5B are, respectively, side view and perspective detailed views of an exemplary embodiment of a peritoneal catheter suitable for use with system of FIG. 1, in which FIG. 5B corresponds to distal region of FIG. 5A.

FIG. 6 is a side view an exemplary bladder catheter suitable for use with the system of FIG. 1.

FIGS. 7A and 7B are, respectively, perspective and top views of the handpiece portion of an exemplary charging and communication system for use in practicing the methods of the present invention;

FIG. 8 is a schematic diagram of the electronic components of an exemplary embodiment of the charging and communication system for use in practicing the methods of the present invention.

FIG. 9 is a schematic diagram of the software implementing the monitoring and control system for use in practicing the methods of the present invention.

FIG. 10 is a screen display of the main screen that is displayed to a physician running monitoring and control software.

FIG. 11 is a schematic view of a first arrangement for instilling DSR fluid into a patient's peritoneal cavity using the system of FIG. 1.

FIG. 12 is a schematic view of an alternative arrangement for instilling DSR fluid into a patient's peritoneal cavity using the system of FIG. 1.

FIGS. 13A and 13B are, respectively, detail views of the connector element of FIG. 12, depicting protuberances for securing the catheters to the connector element and illustrating how the catheter ends may be secured to the connector.

FIG. 14 illustrates steps of an exemplary method in accordance with the principles of the present invention using the system of FIG. 1.

### DETAILED DESCRIPTION OF THE INVENTION

The present invention is directed to system and methods for conducting direct sodium removal (“DSR”) therapy using compositions as described in above-incorporated U.S. Pat. No. 10,918,778 and U.S. patent application Ser. No. 17/661,737. As described in that patent and pending application, DSR therapy may be used to treat fluid overload in various forms of heart failure and renal disease. In accordance with the principles of the present invention, enhanced systems and methods are provided for conducting DSR therapy in an implantable pump is employed to instill DSR solution into the patient's peritoneal cavity, monitor ultrafiltrate and sodium accumulation in the peritoneal cavity and remove such accumulations to the patient's bladder. The enhanced implantable pump may include variable programmable pumping speeds, one or more alarms for alerting the patient of potential pump blockages or malfunction, and selectable options for correcting reported alarm conditions. The implantable pump further may include programming for assessing the progress of the DSR therapy session, and for monitoring pressure fluctuations with the peritoneal cavity, which may serve as triggers for actuating the pump to move sodium-laden DSR solution and ultrafiltrate from the peritoneal cavity to the patient's bladder. In addition, the enhanced implantable pump system may include catheter sets configured to facilitate infusion and removal of DSR infusate into the patient's peritoneal cavity.

As described in the above-incorporate patent and application, the no or low sodium concentration in the DSR solution is configured to cause sodium and fluid (osmotic ultrafiltrate) to pass from the patient's body into the peritoneal cavity. As used in this disclosure, a no or low sodium DSR solution has a sodium content of less than 120 meq/L, more preferably, less than 35 meq/L, and includes infusates having virtually zero concentration of sodium. Accordingly, the methods of the present invention specifically contemplate use of the inventive system to monitor the presence of fluid accumulated in the peritoneal cavity, along with sodium, and to trigger actuation of the pump to move the ultrafiltrate and sodium to the patient's bladder for subsequent voiding via urination.

Exemplary DSR solution formulations presented in the incorporated patent and application include one or more solutions including: D-0.5 to D-50 solutions, i.e., from 0.5 to 50 grams of dextrose per 100 ml of aqueous solution; Icodextrin or dextrin solutions having from 0.5 to 50 grams of icodextrin/dextrin per 100 ml of aqueous solution; urea, and high molecular weight glucose polymer solutions (weight average molecular weight Da>10,000) having from 0.5 to 50 grams of high molecular weight glucose polymer per 100 ml of aqueous solution, and combinations thereof. The aqueous solution includes at least purified water, and may in addition include electrolytes such as low amounts of magnesium or calcium salts, preservatives, ingredients having antimicrobial or antifungal properties, or buffering materials to control pH of the infusate. Icodextrin, a high molecular weight glucose polymer, or other high molecular weight glucose polymer (weight average molecular weight, Da>10,000,) is preferable because it experiences a lower rate of uptake when employed in peritoneal dialysis, and thus lower impact on serum glucose concentrations compared to a dextrose-based solutions.

### Overview of an Exemplary System of the Present Invention

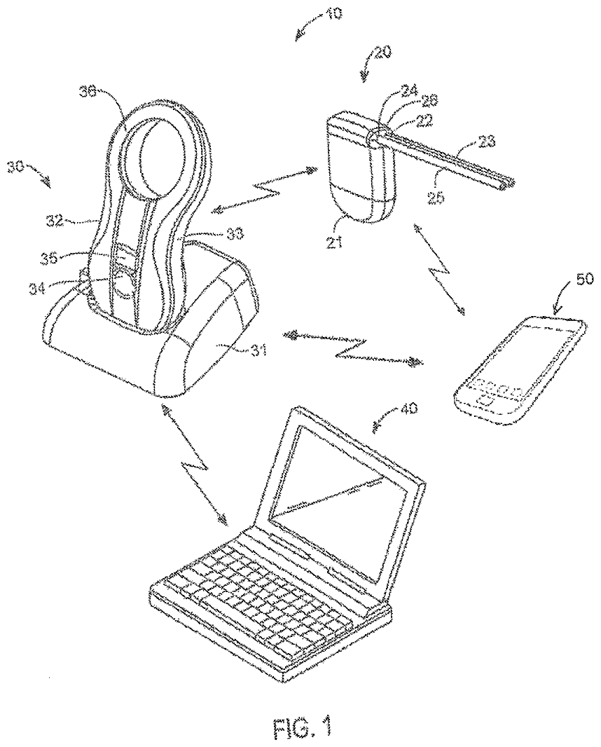

Referring to FIG. 1, exemplary system 10 of the present invention is described. In FIG. 1, components of the system are not depicted to scale on either a relative or absolute basis. System 10 comprises implantable device 20, external charging and communication system 30, software-based monitoring and control system 40, and optionally, smartphone or tablet 50. In the illustrated embodiment, monitoring and control system 40 is configured to be installed and run on a conventional laptop computer, tablet or smartphone, as may be used by the patient's physician. During patient visits, charging and communication system 30 may be coupled, either wirelessly or using a cable, to monitoring and control system 40 to download for review data stored on implantable device 20, or to adjust the operational parameters of the implantable device. Monitoring and control system 40 also may be configured to upload and store date retrieved from charging and communication system 30 to a remote server for later access by the physician or charging and communications system 30. Smartphone or tablet 50 may comprise, for example, the patient's personal smartphone having application software that forms a subset of the capabilities of the software of monitoring and control system 40. In particular, smartphone 50 may include an application configured to communicate with implantable device 20, either directly or through external charging and communication system 30, to present alarms to the patient and permit the patient to issue a limited number of claims directly to the implantable device 20.

Implantable device 20 comprises an electromechanical pump having housing 21 configured for subcutaneous implantation. Implantable device 20 may include an electrically-driven mechanical gear pump and connectors 22 and 24 configured to reduce the risk of improper installation and inadvertent disconnection. Bladder catheter 25 is coupled to pump housing 21 using connector 24. Peritoneal catheter 23 is coupled to pump housing 21 using connector 22. Peritoneal catheter 23 has a proximal end configured to be coupled to pump housing 21 and a distal end configured to be positioned in the peritoneal cavity. Bladder catheter 25 has a proximal end configured to be coupled to pump housing 21 and a distal end configured to be inserted through the wall of, and fixed within, a patient's bladder. In a preferred embodiment, both catheters are made of medical-grade silicone and include polyester cuffs at their distal ends (not shown) to maintain the catheters in position.

In a preferred embodiment, implantable device 20 includes pressure sensors that monitor pressure in one or both of the peritoneal cavity and the bladder. Implantable device 20 may include at least one analyte sensor for generating an output signal corresponding to concentration of a predetermined analyte, such as sodium, or a concentration of an infusate component, such as dextrose or icodextrin. In this manner, movement of sodium-laden DSR solution and ultrafiltrate fluid from the peritoneal cavity to the bladder may be controlled in accordance with one or more target analyte concentrations determined by the physician. In addition, the output of the pressure sensors may cause pumping of fluid into the bladder to be disabled if the bladder pressure reaches a level indicating insufficient space for the bladder to accommodate additional fluid or if the pressure in the peritoneal cavity falls below preset threshold.

In accordance with one aspect of the invention, implantable device 20 may include a variable speed gear pump that may be programmed to pump at different speeds at different times during a DSR therapy session. For example, the implantable pump may be programmed to operate at a first speed to rapidly transfer fluid from a patient's peritoneal cavity to the patient's bladder following completion of a predetermined dwell time or in response to a detected analyte concentration exceeding a predetermined threshold. The implantable pump further may be programmed thereafter to operate at a substantially lower speed to periodically remove further fluid accumulations from the peritoneal cavity during later intervals of, or after the conclusion of, the DSR therapy session. Further, the implantable pump may be operated at a selected speed specifically in response to a command transmitted by the patient, his or her caretaker or physician at specific times during the DSR therapy session or thereafter. Implantable device 20 preferably includes multiple separate fail-safe mechanisms, to ensure that urine cannot pass from the bladder to the peritoneal cavity through the pump, thereby reducing the risk of transmitting infection.

In accordance with another aspect of the invention, the implantable pump system may include an enhanced alarm monitoring and notification capability. In particular, to address the increased pump speed programming complexity, the implantable pump system may include enhanced alarm monitoring, reporting and resolution capability. For example, in one embodiment, the implantable device may include programming for monitoring pump operation and generating an alarm to notify the patient, e.g., via external charging and control system 30 or directly via an application on smartphone 50, that the pump output is below a specified threshold for output for a particular phase of the DSR session. In this case, the patient may be directed to contact his or her physician to request that pump speed be increased for the remainder of the pumping session. Alternatively, if the alarm indicates a blockage in the gear pump, e.g., due to the presence of particulate matter, the alarm transmitted to the patient via smartphone 50 may prompt the patient to activate, or request the physician to activate, a boost mode or jog mode to overcome and/or grind and remove the obstruction, as described below. Still further, the implantable pump may include software that sends an alarm to smartphone 50 if the monitored intraabdominal pressure exceeds a preset threshold during instillation of the DSR fluid or during the dwell period. In this case, the patient may request the physician to cease instillation of further DSR fluid into the peritoneal cavity or to transfer fluid accumulated fluid to the peritoneal cavity to the patient's bladder to resolve the alarm condition. Alternatively, the physician may enable smartphone 50 to permit the patient to make such changes in accordance with the physician's oral directions.

In yet another aspect of the invention, the implantable pump system may include advanced algorithms for determining completion of a dwell period and when to activate the pump to transfer fluid from the peritoneal cavity to the bladder. For example, the above-incorporated patent and application describe that the dwell period for retaining the DSR solution in the peritoneal cavity may be based on a specified time, e.g., 2 hours, attaining a threshold intraabdominal pressure, or attaining an analyte concentration level in the fluid in peritoneal cavity. In accordance with the present invention, actuation of the implantable pump further may be triggered by monitoring the rate of change of pressure within the peritoneal cavity over a specified time interval. In this manner, the pump will not be inadvertently triggered by transient fluctuations in intraabdominal pressure, e.g., due to patient coughing or transient postural changes. Instead, the implantable pump will begin transferring fluid from the peritoneal cavity to the bladder only after a sustained increase in pressure exceeding a predetermined threshold is detected.

Still referring to FIG. 1, external charging and communication system 30 of the exemplary system, preferably includes base 31 and handpiece 32. Handpiece 32 may contain a controller, a radio transceiver, an inductive charging circuit, a battery, a quality-of-charging indicator and a display, and may be removably coupled to base 31 to recharge the battery. Base 31 may contain a transformer and circuitry for converting conventional 120V or 220-240V service to a suitable DC current to charge handpiece 32 when coupled to base 31. Alternatively, handpiece 32 may include such circuitry and a detachable power cord, thus permitting the handpiece to be directly plugged into a wall socket to charge the battery. Preferably, each of implantable device 20 and handpiece 32 includes a device identifier stored in memory, such that handpiece 32 provided to the patient is coded to operate only with that patient's specific implantable device 20.

Handpiece 32 illustratively includes housing 33 having multi-function button 34, display 35, a plurality of light emitting diodes (LEDs, not shown) and inductive coil portion 36. Multi-function button 34 enables the patient to issue a limited number of commands to implantable device 20, while display 35 provides visible confirmation that a desired command has been input; it also may display battery status of implantable device 20. Inductive coil portion 36 houses an inductive coil that is used transfer energy from handpiece 32 to recharge the battery of implantable device 20. The LEDs, which are visible through the material of housing 33 when lit, may be arranged in three rows of two LEDs each, and are coupled to the control circuitry and inductive charging circuit contained within handpiece 32. The LEDs may be arranged to light up to reflect the degree of inductive coupling achieved between handpiece 32 and implantable device 20 during recharging of the latter. Alternatively, the LEDs may be omitted and an analog display provided on display 35 indicating the quality of inductive coupling.

Control circuitry contained within handpiece 32 is coupled to the inductive charging circuit, battery, LEDs and radio transceiver, and includes memory for storing information received from implantable device 20. Handpiece 32 also preferably includes a data port, such as a USB port, that permits the handpiece to be coupled to monitoring and control system 40 during visits by the patient to the physician's office. Alternatively, handpiece 32 may include a wireless chip, e.g., that conforms to the Bluetooth or IEEE 802.11 wireless standards, thereby enabling the handpiece to communicate wirelessly with monitoring and control system 40, either directly or via the Internet.

External charging and communication system 30 also may be configured to communicate directly with smartphone 50, as well as monitoring and control system 40. Thus, implantable device 20 may be configured to communicate with smartphone 50 via external charging and control system 30 when external charging and communication system 40 is coupled to implantable device 20, for example, when charging. Implantable device 20 also may be configured directly with smartphone 50, for example, to communicate alarms to smartphone 50 and to receive commands to resolve such alarms from smartphone 50. Smartphone 50 also may communicate alarm conditions, and the resolution of such conditions, to external charging and communication system 30 for storage and later reporting to monitoring and control system 40.

Monitoring and control system 40 is intended primarily for use by the physician and comprises software configured to run on a conventional computer, e.g., a laptop as illustrated in FIG. 1, or a tablet or smartphone. The software enables the physician to configure, monitor and control operation of charging and communication system 30 and implantable device 20. The software may include routines for configuring and controlling pump operation, such as a target analyte concentration at which pumping is actuated, the amount of analyte transferred from the peritoneal cavity to the bladder during a pumping session, pumping speeds for various intervals of the dwell period and post-dwell period, a maximum dwell time for the DSR therapy, and limits on intraabdominal pressure, bladder pressure, pump pressure, and battery temperature. System 40 also may provide instructions to implantable device 20 via charging and control system 30 to control operation of implantable device 20 so as not to move fluid during specific periods (e.g., at night or when the bladder is full) or to defer pump actuation.

System 40 also may be configured, for example, to send immediate commands to the implantable device to start or stop the pump, or to operate the pump in reverse or at high power to unblock the pump or associated catheters, a subset of which may be available to the patient using an application installed on smartphone 50. The software of system 40 may be configured to download real-time data relating to pump operation, as well as event logs stored during operation of implantable device 20. Based on the downloaded data, e.g., based on measurements made of the patient's analyte concentration, intraabdominal pressure, respiratory rate, and/or fluid accumulation, the software of system 40 optionally may be configured to analyze such data to alert the physician to the development of potential trends. Such analyses may include generating alerts regarding prediction or detection of heart failure decompensation or a change in the patient's health for which an adjustment to the flow rate, volume, time and/or frequency of pump operation may be required. Finally, system 40 optionally may be configured to remotely receive raw or filtered operational data from handpiece 32 over a secure Internet channel.

### The Implantable Device

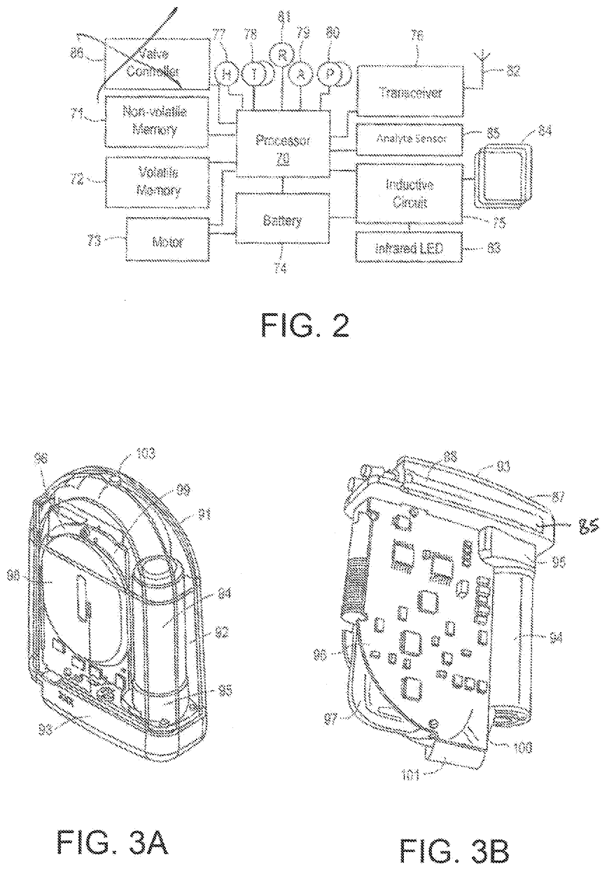

Referring now to FIG. 2, a schematic depicting the functional blocks of implantable device 20 suitable for use in practicing the methods of the present invention is described. Implantable device 20 includes control circuitry, illustratively processor 70 coupled to nonvolatile memory 71, such as flash memory or electrically erasable programmable read only memory, and volatile memory 72 via data buses. Processor 70 is electrically coupled to electric motor 73, battery 74, inductive circuit 75, radio transceiver 76, one or more analyte sensors 85, and a plurality of other sensors, including humidity sensor 77, one or more temperature sensors 78, accelerometer 79, pressure sensors 80, and respiratory rate sensor 81. Inductive circuit 75 is electrically coupled to coil 84 to receive energy transmitted from charging and communication system 30, while transceiver 76 is coupled to antenna 82, and likewise is configured to communicate with a transceiver in charging and communication system 30 and smartphone 50, as described below. Optionally, inductive circuit 75 also may be coupled to infrared light emitting diode 83. Motor 73 may include a dedicated controller, which interprets and actuates motor 73 responsive to commands from processor 70. All of the components depicted in FIG. 2 are contained within a low volume sealed biocompatible housing, depicted in FIG. 3A.

Processor 70 executes firmware stored in nonvolatile memory 71 which controls operation of motor 73 responsive to signals generated by motor 73, sensors 77-81 and 85, and commands received from transceiver 76. Processor 70 also controls reception and transmission of messages via transceiver 76 and operation of inductive circuit 75 to charge battery 74. In addition, processor 70 receives signals generated by Hall Effect sensors located within motor 73, which are used to compute direction and revolutions of the gears of the gear pump, and thus fluid volume transferred and the viscosity of that fluid, as described below. Processor 70 preferably includes a low-power mode of operation and includes an internal clock, such that the processor can be periodically awakened to handle pumping, pump tick mode, or communications and charging functions, and/or awakened to handle commands received by transceiver 76 from handpiece 32. In one embodiment, processor 70 comprises a member of the MSP430 family of microcontroller units available from Texas Instruments, Incorporated, Dallas, Tex., and may incorporate the nonvolatile memory, volatile memory, and radio transceiver components depicted in FIG. 2. In addition, the firmware executed on processor 70 may be configured to respond directly to commands sent to implantable device 20 via charging and communication system 30. Processor 70 also is configured to monitor operation of motor 72 (and any associated motor controller) and sensors 77-81 and 85, as described below, and to store data reflecting operation of the implantable device, including event logs, and to generate alarms corresponding to various fault conditions. Such stored data and alarms may be reported to the charging and communication system when it is next wirelessly coupled to the implantable device, while alarms also may be communicated to smartphone 50. In a preferred embodiment, processor 70 generates up to eighty log entries per second prior to activating the pump, about eight log entries per second when the implantable system is actively transferring fluid and about one log entry per hour when not transferring fluid. As discussed above, processor 70 may be programmed to operate motor 73 at variable speeds responsive to stored programming or commands received directly via external charging and communication system 30 or smartphone 50.

Nonvolatile memory 71 preferably comprises flash memory or EEPROM, and stores a unique device identifier for implantable device 20, firmware to be executed on processor 70, configuration set point data relating to operation of the implantable device, and optionally, coding to be executed on transceiver 76 and/or inductive circuit 75, and a separate motor controller, if present. Firmware and set point data stored on nonvolatile memory 71 may be updated using new instructions provided by control and monitoring system 40 via charging and communication system 30. Volatile memory 72 is coupled to and supports operation of processor 70, and stores data and event log information gathered during operation of implantable device 20. Volatile memory 72 also serves as a buffer for communications sent to, and received from, charging and communication system 30 and smartphone 50.

Transceiver 76 preferably comprises a radio frequency transceiver and is configured for bi-directional communications via antenna 76 with a similar transceiver circuit disposed in handpiece 32 of charging and communication system 30. Transceiver 76 may include a communications circuit, e.g., near field or Bluetooth, for directly communicating in a bi-directional manner with an application program loaded on smartphone 50. Transceiver 76 also may include a low power mode of operation, such that it periodically awakens to listen for incoming messages and responds only to those messages that include the unique device identifier assigned to that implantable device. Alternatively, transceiver 76 may be configured to send or receive data to external charging and communications system 30 only when inductive circuit 75 of the implantable device is active. Transceiver 76 may employ an encryption routine to ensure that messages sent from, or received by, the implantable device cannot be intercepted or forged.

Inductive circuit 75 is coupled to coil 84, and is configured to recharge battery 74 of the implantable device when exposed to a magnetic field applied by a corresponding inductive circuit within handpiece 32 of charging and communication system 30. In one embodiment, inductive circuit 75 is coupled to optional infrared LED 83 that emits an infrared signal when inductive circuit 75 is active. The infrared signal may be received by handpiece 32 to assist in locating the handpiece relative to the implantable device, thereby improving the magnetic coupling and energy transmission.

Inductive circuit 75 optionally may be configured not only to recharge battery 74, but to directly provide energy to motor 73 in a “boost” mode or jog/shake mode to unblock the pump, for example, in response to a command issued by smartphone 50. In particular, if processor 70 detects that motor 73 is stalled, e.g., due to a block created by fibrin or other debris in the peritoneal cavity, an alarm may be generated and sent to smartphone 50. When the alarm is reported to smartphone 50, and the patient may issue a command to processor 70 to apply an overvoltage to motor 73 from inductive circuit 75 for a predetermined time period to free the pump blockage. Alternatively, depressing the patient command may cause processor 70 to execute a set of commands by which motor 73 is jogged or shaken, e.g., by alternatingly running the motor in reverse and then forward, to disrupt the blockage. Because such modes of operation may employ higher energy consumption than expected during normal operation, it is advantageous to drive the motor during such procedures with energy supplied via inductive circuit 75.

Battery 74 preferably comprises a lithium ion or lithium polymer battery capable of long lasting operation, e.g., up to three years, when implanted in a human, so as to minimize the need for re-operations to replace implantable device 20. In one preferred embodiment, battery 74 supplies a nominal voltage of 3.6V, a capacity of 150 mAh when new, and a capacity of about 120 mAh after two years of use. Preferably, battery 74 is configured to supply a current of 280 mA to motor 73 when pumping; 25 mA when the transceiver is communicating with charging and communication system 30; 8 mA when processor 70 and related circuitry is active, but not pumping or communicating; and 0.3 mA when the implantable device is in low power mode. More preferably, battery 74 should be sized to permit a minimum current of at least 450 mAh for a period of 10 seconds and 1 A for 25 milliseconds during each charging cycle.

Motor 73 preferably is a brushless direct current or electronically commuted motor having a splined output shaft that drives a set of floating gears that operate as a gear pump, as described below. Motor 73 may include a dedicated motor controller, separate from processor 70, for controlling operation of the motor, and preferably is capable of operating at different speeds to achieve different fluid transfer rates. Motor 73 may include a plurality of Hall Effect sensors, preferably two or more, for determining motor position and direction of rotation. Due to the high humidity that may be encountered in implantable device 20, processor 70 may include programming to operate motor 73, although with reduced accuracy, even if some or all of the Hall Effect sensors fail.

In a preferred embodiment, motor 73 is capable of driving the gear pump to generate a nominal flow rate of 150 ml/min and applying a torque of about 1 mNm against a pressure head of 30 cm water at 3000 RPM. In this embodiment, the motor preferably is selected to drive the gears at from 1000 to 5000 RPM, corresponding to flow rates of from 50 to 260 ml/min, respectively. The motor preferably has a stall torque of at least 3 mNm at 500 mA at 3 V, and more preferably 6 mNm in order to crush non-solid proteinaceous materials. As discussed above, the motor preferably also supports a boost mode of operation, e.g., at 5 V, when powered directly through inductive circuit 75. Motor 73 preferably also is capable of being driven in reverse as part of a jogging or shaking procedure to unblock the gear pump.

Processor 70 also may be programmed to automatically and periodically wake up and enter a pump tick mode. In this mode of operation, the gear pump is advanced slightly, e.g., about 120 degrees as measured by the Hall Effect sensors, before processor 70 returns to low power mode. Preferably, this interval is about every 20 minutes, although it may be adjusted by the physician using the monitoring and control system. Tick mode is expected to prevent the DSR solution and ultrafiltrate from partially solidifying and blocking the gear pump.

Processor 70 also may be programmed to enter a jog or shake mode when operating on battery power alone, to unblock the gear pump, upon receipt of a command from smartphone 50 or charging and communication system 30. Similar to the boost mode available when charging the implantable device with the handpiece of charging and communication system 30, the jog or shake mode causes the motor to rapidly alternate the gears between forward and reverse directions to crush or loosen any buildup of tissue or other debris in the gear pump or elsewhere in the fluid path. Specifically, in this mode of operation, if the motor does not start to turn within a certain time period after it is energized (e.g., 1 second), the direction of the motion is reversed for a short period of time and then reversed again to let the motor turn in the desired direction. If the motor does still not turn (e.g., because the gear pump is jammed) the direction is again reversed for a period of time (e.g., another 10 msec). If the motor still is unable to advance, the time interval between reversals of the motor direction is reduced to allow the motor to develop more power, resulting in a shaking motion of the gears. If the motor does not turn forward for more than 4 seconds, the jog mode of operation ceases, and an alarm is generated and also written to the event log. If the motor was unable to turn forward, processor 70 may introduce a backwards tick before the next scheduled fluid movement. A backward tick is the same as a tick (e.g., about 120 degrees forward movement of the motor shaft) but in a reverse direction, and is intended to force the motor backwards before turning forward, thus allowing the motor to gain momentum.

Sensors 77-81 continually monitor humidity, temperature, acceleration, pressure, and respiratory rate, and provide corresponding signals to processor 70 which stores the corresponding data in memory 71 for later transmission to monitoring and control system 40. In particular, humidity sensor 77 is arranged to measure humidity within the housing of the implantable device, to ensure that the components of implantable device are operated within expected operational limits. Humidity sensor 77 preferably is capable of sensing and reporting humidity within a range or 20% to 100% with high accuracy. One or more of temperature sensors 78 may be disposed within the housing and monitor the temperature of the implantable device, and in particular battery 74 to ensure that the battery does not overheat during charging, while another one or more of temperature sensors 78 may be disposed so as to contact fluid entering at inlet 62 and thus monitor the temperature of the fluid, e.g., for use in assessing the patient's health. Accelerometer 79 is arranged to measure acceleration of the implant, preferably along at least two axes, to detect periods of activity and inactivity, e.g., to determine whether the patient is sleeping or to determine whether and when the patient is active. This information is provided to processor 70 to ensure that the pump is not operated when the patient is indisposed to attend to voiding of the bladder.

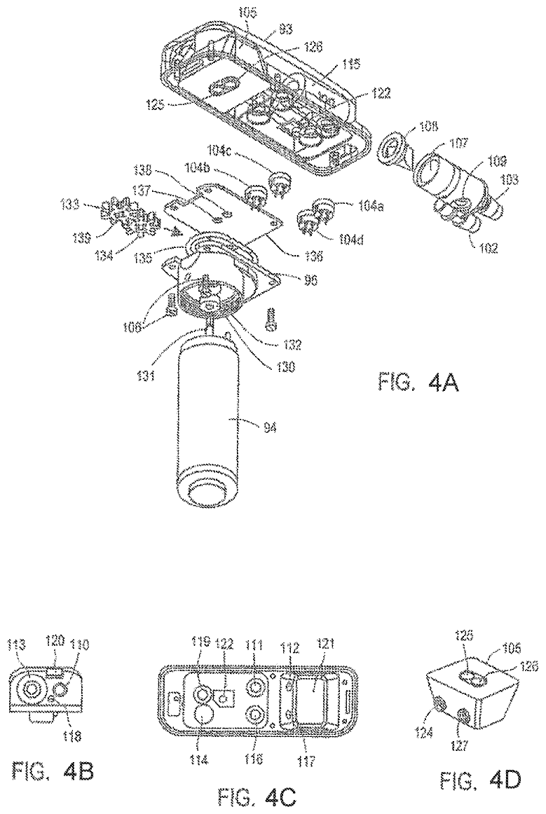

Implantable device 20 preferably includes multiple pressure sensors 80, which are continually monitored during waking periods of the processor. As described below with respect to FIG. 4A, the implantable device preferably includes four pressure sensors: a sensor to measure the pressure in the peritoneal cavity, a sensor to measure the ambient pressure, a sensor to measure the pressure at the outlet of the gear pump, and a sensor to measure the pressure in the bladder. These sensors preferably are configured to measure absolute pressure between 450 mBar and 1300 mBar while consuming less than 50 mW at 3V. Preferably, the sensors that measure pressure at the pump outlet and in the bladder are placed across a duckbill valve, which prevents reverse flow of urine and/or used DSR solution and ultrafiltrate back into the gear pump and also permits computation of flow rate based on the pressure drop across the duckbill valve.

Respiratory rate monitor 81 is configured to measure the patient's respiratory rate, e.g., for use in assessing the patient's health. Alternatively, the patient's respiratory rate may be measured based on the outputs of one or more of pressure sensors 80, e.g., based on changes in the ambient pressure or the pressure in the peritoneal cavity caused by the diaphragm periodically compressing that cavity during breathing.

In accordance with one aspect of the present invention, analyte sensor 85 may be a chemical or biochemical sensor configured to monitor a sodium concentration of sodium-laden DSR solution and ultrafiltrate instilled into and accumulated within the patient's peritoneal cavity. Analyte sensor 85 may in addition sense a concentration of a component, such as dextrose or icodextrin concentration, in the DSR solution present in the peritoneal cavity, and that information may be used by the processor of implantable device 20 to trigger starting or stopping of the pump. One exemplary in vivo sensor suitable for monitoring sodium ion concentration is described in U.S. Patent Application Publication No. 2008/033260, the entirety of which is incorporated herein by reference. Analyte sensor 85 may be disposed on catheter 23, on catheter 25, or may be disposed within the housing of implantable device 20 so as to contact fluid flowing through the device. Any desired number of additional sensors for measuring the health of the patient also may be provided in operable communication with processor 70 and may output recordable parameters for storage in memory 71 and transmission to monitoring and control system 40, that the physician may use to assess the patient's health.

In an exemplary embodiment, processor 70 may be programmed to monitor an output of analyte sensor 85, for example, indicative of a value of sodium concentration, and to compare that value to a target sodium concentration value selected by the physician at which implantable device 20 is activated to transfer fluid from the peritoneal cavity to the patient's bladder. Alternatively, or in addition, analyte sensor 85 could measure a concentration of an infusate component remaining in the peritoneal cavity. Processor 70 also may be programmed to activate implantable device 20 to move fluid from the peritoneal cavity to the bladder after that fluid has dwelled in the peritoneal cavity a sufficient period that the measured analyte concentration exceeds or falls below a target value. In addition, fluid transfer may be initiated after infusate has dwelled in the peritoneal cavity for a predetermined maximal amount of time, which may be set by a physician. For this purpose, processor 70 may include a programmed timer that monitors the dwell time, e.g., elapsed time from when the DSR solution is first introduced into the patient's peritoneal cavity. As a further alternative, processor 70 may be programmed to compute an estimate of the amount of analyte transferred from the peritoneal cavity to the bladder as function of the measured concentration and the volume of fluid transferred, and to report that amount to the patient, physician or caretaker. Such information may, in turn, be used to determine when the next DSR therapy session should occur or be used in adjusting the volume of DSR solution to be employed during a subsequent DSR therapy session.

The volume of fluid transferred, and pump activation time and frequency may be selected to optimize sodium removal to maintain or improve the patient's health, to alleviate the fluid overload and to ensure a stable serum sodium level. These parameters may be selected based on the patient's symptoms, the activity and habits of the patient, the permeability of the peritoneal membrane and the osmotic characteristics of the DSR solution, and they may be static or changed by the physician on a session-by-session basis. For example, the physician may initially program processor 70 with a first sodium concentration level, amount of sodium to be removed, maximum dwell time, volume, or frequency based on his perception of the patient's health and habits, and later adjust that initial programming to vary those parameters based on his perception of changes in the patient's health, for example based on changes over time in parameters measured by implantable device 20 and relayed to the physician via monitoring and control software 40.

Processor 70 also may be programmed to monitor the sensors 77-81 and to generate an alarm condition that is relayed to the patient and/or the clinician indicative of a potential decline in the patient's health. For example, processor 70 may monitor pressure sensors 80 to determine whether, over predetermined time intervals, there is an increase in pressure within the peritoneal cavity. Such pressure increases may be the result of an increase in the rate of accumulation of fluid in the peritoneal cavity, which may in turn indicate heart failure decompensation. Such alarm condition may inform the patient to seek immediate treatment.

Processor 70 further may be programmed to pump fluid from the peritoneal cavity to the bladder only when the pressure in the peritoneal cavity exceeds a first predetermined value, and the pressure in the bladder is less than a second predetermined value, so that the bladder does not become overfull. To account for patient travel from a location at sea level to a higher altitude, the ambient pressure measurement may be used to calculate a differential value for the peritoneal pressure. In this way, the predetermined pressure at which the pump begins operation may be reduced, to account for lower atmospheric pressure. Likewise, the ambient pressure may be used to adjust the predetermined value for bladder pressure. In this way, the threshold pressure at which the pumping ceases may be reduced, because the patient may experience discomfort at a lower bladder pressure when at a high altitude location.

As yet another alternative, processor 70 may be programmed to monitor intraabdominal pressure over a specified interval, e.g., one to three minutes or more, and to activate the implantable pump only when the pressure exceeds a specified threshold value for the specified interval. In particular, because the abdominal cavity is compliant, it may be desirable to monitor and trigger pump actuation based on the time-dependent evolution of pressure within the peritoneal cavity. In this manner, the implantable pump could differentiate between short-duration pressure fluctuations, e.g., caused by the patient coughing or bending over, from pressure increases resulting from filling of the peritoneal cavity due to ultrafiltrate accumulation.

Further, processor 70 may be programmed to permit transfer of fluid from the peritoneal cavity to the bladder to be interrupted, for example if the bladder pressure indicates that the bladder is full, and to resume transferring fluid once the bladder pressure sensor detects that the bladder has been voided. In this case, because the sodium concentration level may fall due to fluid transfer immediately before the bladder was detected to be full, the processor may be programmed to resume fluid transfer once the sodium concentration level has reached the target level during a single DSR therapy session.

Optionally, implantable device 20 may include a UV lamp (not shown) disposed in operable communication with controller 70. The UV lamp may be configured to irradiate and thus kill pathogens in the DSR solution before is instilled into and/or after fluid is extracted from the peritoneal cavity. The UV lamp preferably generates light in the UV-C spectral range (about 200-280 nm), particularly in the range of about 250-265 nm, which is also referred to as the “germicidal spectrum” because light in that spectral range breaks down nucleic acids in the DNA of microorganisms. Low-pressure mercury lamps have an emission peak at approximately 253.7 nm, and may suitably be used for UV lamp 85. Alternatively, the UV lamp may be a UV light emitting diode (LED), which may be based on AlGaAs or GaN.

Referring now to FIGS. 3A and 3B, further details of an exemplary embodiment of implantable device 20 are provided. In FIG. 3A, housing 91 appears transparent, although it should of course be understood that housing 91 comprises an opaque biocompatible plastic, glass and/or metal alloy materials. In FIG. 3B, the implantable device is shown with lower portion 92 of housing 91 removed from upper housing 93 and without a glass bead/epoxy filler material that is used to prevent moisture from accumulating in the device. In FIGS. 3A and 3B, motor 94 is coupled to gear pump housing 95, which is described in greater detail with respect to FIG. 4A. The electronic components discussed above with respect to FIG. 2 are disposed on circuit board substrate 96, which extends around and is fastened to support member 97. Coil 98 (corresponding to coil 84 of FIG. 2) is disposed on flap 99 of the substrate and is coupled to the electronic components on flap 100 by flexible cable portion 101. Support member 97 is fastened to upper housing 93 and provides a cavity that holds battery 102 (corresponding to battery 74 of FIG. 2). Lower portion 92 of housing 91 includes port 103 for injecting the glass bead/epoxy mixture after upper portion 93 and lower portion 92 of housing 91 are fastened together, to reduce space in the housing in which moisture can accumulate.

Housing 91 also may include features designed to reduce movement of the implantable pump once implanted within a patient, such as a suture hole to securely anchor the implantable device to the surrounding tissue. Housing 91 may in addition include a polyester ingrowth patch that facilitates attachment of the implantable device to the surrounding tissue following subcutaneous implantation.

Additionally, the implantable device optionally may incorporate anti-clogging agents, such enzyme eluting materials that specifically target the proteinaceous components of fluid from the peritoneal cavity, enzyme eluting materials that specifically target the proteinaceous and encrustation promoting components of urine, chemical eluting surfaces, coatings that prevent adhesion of proteinaceous compounds, and combinations thereof. Such agents, if provided, may be integrated within or coated upon the surfaces of the various components of the system.

Referring now to FIGS. 4A to 4D, further details of the gear pump and fluid path are described. In FIGS. 4A-4D, like components are identified using the same reference numbers from FIGS. 3A and 3B. FIG. 4A is an exploded view showing assembly of motor 94 with gear pump housing 95 and upper housing 93, as well as the components of the fluid path within the implantable device. Upper housing 93 preferably comprises a high strength plastic or metal alloy material that can be molded or machined to include openings and channels to accommodate inlet nipple 102, outlet nipple 103, pressure sensors 104a-104d, manifold 105 and screws 106. Nipples 102 and 103 preferably are machined from a high strength biocompatible metal alloy, and outlet nipple 103 further includes channel 107 that accepts elastomeric duckbill valve 108. Outlet nipple 103 further includes lateral recess 109 that accepts pressure sensor 104a, which is arranged to measure pressure at the inlet end of the bladder catheter, corresponding to pressure in the patient's bladder (or peritoneal cavity).

Referring now also to FIGS. 4B and 4C, inlet nipple 102 is disposed within opening 110, which forms a channel in upper housing 93 that includes opening 111 for pressure sensor 104b and opening 112 that couples to manifold 105. Pressure sensor 104b is arranged to measure the pressure at the outlet end of the peritoneal catheter, corresponding to pressure in the peritoneal cavity. Outlet nipple 103, including duckbill valve 107, is disposed within opening 113 of upper housing 93 so that lateral recess 108 is aligned with opening 114 to permit access to the electrical contacts of pressure sensor 104a. Opening 113 forms channel 115 that includes opening 116 for pressure sensor 104c, and opening 117 that couples to manifold 105. Upper housing 93 preferably further includes opening 118 that forms a channel including opening 119 for accepting pressure sensor 104d. Pressure sensor 104d measures ambient pressure, and the output of this sensor is used to calculate differential pressures as described above. Upper housing further includes notch 120 for accepting connector 26 (see FIG. 4B) for retaining the peritoneal and bladder catheters coupled to inlet and outlet nipples 102 and 103. Upper housing 93 further includes recess 121 to accept manifold 105, and peg 122, to which support member 97 (see FIG. 3B) is connected.

Manifold 105 preferably comprises a molded elastomeric component having two separate fluid channels (such channels designated 88 in FIG. 3B) that couple inlet and outlet flow paths through upper housing 93 to the gear pump. The first channel includes inlet 124 and outlet 125, while the second channel includes inlet 126 and outlet 127. Inlet 124 couples to opening 112 (see FIG. 4C) of the peritoneal path and outlet 127 couples to opening 117 of the bladder path. Analyte sensor 85 (see FIG. 3B) may be located in communication with the peritoneal fluid path, or optionally located on catheter 23. Manifold 105 is configured to improve manufacturability of the implantable device, by simplifying construction of upper housing 93 and obviating the need to either cast or machine components with complicated non-linear flow paths.

Motor 94 is coupled to gear pump housing 95 using mating threads 130, such that splined shaft 131 of motor 94 passes through bearing 132. The gear pump of the present invention comprises intermeshing gears 133 and 134 enclosed in gear pump housing 95 by O-ring seal 135 and plate 136. The gear pump is self-priming. Plate 136 includes openings 137 and 138 that mate with outlet 125 and inlet 126 of manifold 105, respectively. Splined shaft 131 of motor 94 extends into opening 139 of gear 133 to provide floating engagement with that gear.

### Peritoneal and Bladder Catheters

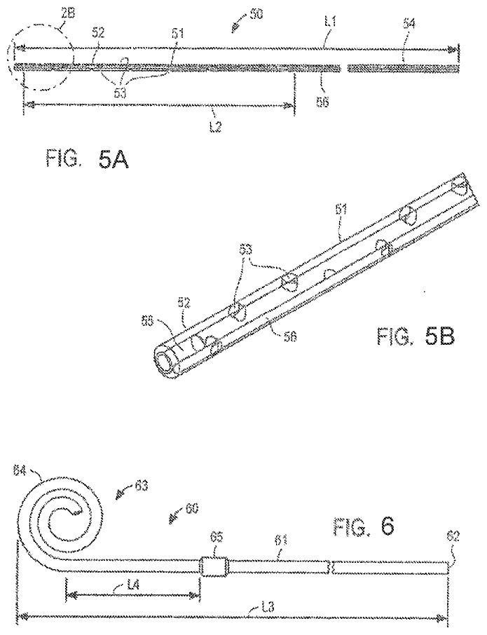

Referring to FIGS. 5A and 5B, peritoneal catheter 50 may be Medionics International Inc.'s peritoneal dialysis Catheter, Model No. PSNA-100 or a catheter having similar structure and functionality. Peritoneal catheter 50 corresponds to peritoneal catheter 23 of FIG. 1, and may comprise tube 51 of medical-grade silicone including inlet (distal) end 52 having a plurality of through-wall holes 53 and outlet (proximal) end 54. Holes 53 may be arranged circumferentially offset by about 90 degrees, as shown in FIG. 5B. Peritoneal catheter 50 may also include a polyester cuff (not shown) in the region away from holes 53, to promote adhesion of the catheter to the surrounding tissue, thereby anchoring it in place. Alternatively, inlet end 52 of peritoneal catheter 50 may have a spiral configuration, and an atraumatic tip, with holes 53 distributed over a length of the tubing to reduce the risk of clogging.

Inlet end 52 also may include a polyester cuff to promote adhesion of the catheter to an adjacent tissue wall, thereby ensuring that the inlet end of the catheter remains in position. Outlet end 54 also may include a connector for securing the outlet end of the peritoneal catheter to implantable device 20. In one preferred embodiment, the distal end of the peritoneal catheter, up to the ingrowth cuff, may be configured to pass through a conventional 16 F peel-away sheath. In addition, the length of the peritoneal catheter may be selected to ensure that it lies along the bottom of the body cavity, and is sufficiently resistant to torsional motion so as not to become twisted or kinked during or after implantation.

With respect to FIG. 6, an exemplary embodiment of bladder catheter 60 is described, corresponding to bladder catheter 25 of FIG. 1. Bladder catheter 60 preferably comprises tube 61 of medical-grade silicone having inlet (proximal) end 62 and outlet (distal) end 63 including spiral structure 64, and polyester ingrowth cuff 65. Bladder catheter 60 includes a single internal lumen that extends from inlet end 62 to a single outlet at the tip of spiral structure 64, commonly referred to as a “pigtail” design. Inlet end 62 may include a connector for securing the inlet end of the bladder catheter to implantable device 20, or may have a length that can be trimmed to fit a particular patient. In one embodiment, bladder catheter 60 may have length L3 of about 45 cm, with cuff 65 placed length L4 of about 5 to 6 cm from spiral structure 64. Bladder catheter 60 may be loaded onto a stylet with spiral structure 64 straightened, and implanted using a minimally invasive technique in which outlet end 63 and spiral structure 64 are passed through the wall of a patient's bladder using the stylet. When the stylet is removed, spiral structure 64 returns to the coiled shape shown in FIG. 6. Once outlet end 63 of bladder catheter 60 is disposed within the patient's bladder, the remainder of the catheter is implanted using a tunneling technique, such that inlet end 62 of the catheter may be coupled to implantable device 20. Spiral structure 64 may reduce the risk that outlet end 63 accidentally will be pulled out of the bladder before the tissue surrounding the bladder heals sufficiently to incorporate ingrowth cuff 65, thereby anchoring the bladder catheter in place.

In a preferred embodiment, bladder catheter 60 is configured to pass through a conventional peel-away sheath. Bladder catheter 60 preferably is sufficiently resistant to torsional motion so as not to become twisted or kinked during or after implantation. In a preferred embodiment, peritoneal catheter 50 and bladder catheter 60 preferably are different colors, have different exterior shapes (e.g., square and round) or have different connection characteristics so that they cannot be inadvertently interchanged during connection to implantable device 20. Optionally, bladder catheter 60 may include an internal duckbill valve positioned midway between inlet 62 and outlet end 63 of the catheter to ensure that urine does not flow from the bladder into the peritoneal cavity if the bladder catheter is accidentally pulled free from the pump connector of implantable device 20.

In an alternative embodiment, the peritoneal and bladder catheters devices may incorporate one or several anti-infective agents to inhibit the spread of infection between body cavities. Examples of anti-infective agents which may be utilized may include, e.g., bacteriostatic materials, bactericidal materials, one or more antibiotic dispensers, antibiotic eluting materials, and coatings that prevent bacterial adhesion, and combinations thereof. Additionally, implantable device 20 may include a UV lamp configured to irradiate fluid in the peritoneal and/or bladder catheters so as to kill any pathogens that may be present and thus inhibit the development of infection.

Alternatively, rather than comprising separate catheters, peritoneal and bladder catheters 50, 60 may share a common wall as depicted in FIG. 1, which may be convenient to facilitate insertion of a single dual-lumen tube. In addition, either or both of the peritoneal or bladder catheters may be reinforced along a portion of its length or along its entire length using ribbon or wire braiding or lengths of wire or ribbon embedded or integrated within or along the catheters. The braiding or wire may be fabricated from metals such as stainless steels, superelastic metals such as nitinol, or from a variety of suitable polymers. Such reinforcement may also be used for catheter 46 connected to optional reservoir 45.

### The Charging and Communication System

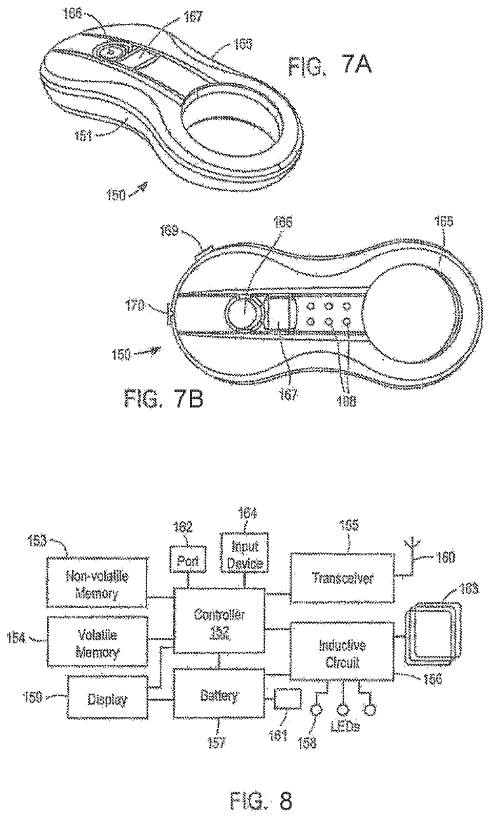

Referring to FIGS. 7A, 7B and 8, charging and communication system 150 (corresponding to system 30 of FIG. 1) is described in greater detail. In one preferred embodiment, charging and communication system 150 comprises handpiece 151 and base 31 (see FIG. 1). Base 31 provides comprises a cradle for recharging handpiece 151, and preferably contains a transformer and circuitry for converting conventional 120/220/240V power service to a suitable DC current to charge handpiece 151 when it is coupled to the base. Alternatively, handpiece 151 may include circuitry for charging the handpiece battery, and a detachable power cord. In this embodiment, handpiece 151 may be plugged into a wall socket for charging, and the power cord removed when the handpiece is used to recharge the implantable device.

As shown in FIG. 8, handpiece 151 contains controller 152, illustratively the processor of a micro-controller unit coupled to nonvolatile memory 153 (e.g., either EEPROM or flash memory), volatile memory 154, radio transceiver 155, inductive circuit 156, battery 157, indicator 158 and display 159. Controller 152, memories 153 and 154, and radio transceiver 155 may be incorporated into a single microcontroller unit, such as the MPS430 family of microprocessors, available from Texas Instruments Incorporated, Dallas, Tex. Transceiver 155 is coupled to antenna 160 for sending and receiving information to implantable device 20. Battery 157 is coupled to connector 161 that removably couples with a connector in base 31 to recharge the battery. Port 162, such as a USB port or comparable wireless circuit, is coupled to controller 152 to permit information to be exchanged between handpiece 151 and the monitoring and control system. Inductive circuit 156 is coupled to coil 163. Input device 164, preferably a multi-function button, also is coupled to controller 152 to enable a patient to input a limited number of commands. Indicator 158 illustratively comprises a plurality of LEDs that illuminate to indicate the quality of charge coupling achieved between the handpiece and implantable device, and therefore assist in optimizing the positioning of handpiece 151 relative to the implantable device during recharging. In one preferred embodiment, indicator 158 is omitted, and instead a bar indicator provided on display 159 that indicates the quality-of-charging resulting from the coupling of coils 163 and 84.

In a preferred embodiment, handpiece 151 includes a device identifier stored in nonvolatile memory 153 that corresponds to the device identifier stored in nonvolatile memory 71 of the implantable device, such that handpiece 151 will communicate only with its corresponding implantable device 20. Optionally, a configurable handpiece for use in a physician's office may include the ability to interrogate an implantable device to request that device's unique device identifier, and then change the device identifier of the monitoring and control system 40 to that of the patient's implantable device, so as to mimic the patient's handpiece. In this way, a physician may adjust the configuration of the implantable device if the patient forgets to bring his handpiece 151 with him during a visit to the physician's office.

Controller 152 executes firmware stored in nonvolatile memory 153 that controls communications and charging of the implantable device. Controller 152 also is configured to transfer and store data, such as event logs, uploaded to handpiece 151 from the implantable device, for later retransmission to monitoring and control system 40 via port 162, during physician office visits. Alternatively, handpiece 151 may be configured to recognize a designated wireless access point within the physician's office, and to wirelessly communicate with monitoring and control system 40 during office visits. As a further alternative, base 31 may include telephone circuitry for automatically dialing and uploading information stored on handpiece 151 to a physician's website via a secure connection, such as alarm information.

Controller 152 preferably includes a low-power mode of operation and includes an internal clock, such that the controller periodically awakens to communicate with the implantable device to log data or to perform charging functions. Controller 152 preferably is configured to awaken when placed in proximity to the implantable device to perform communications and charging functions, and to transmit commands input using input device 164. Controller 152 further may include programming for evaluating information received from the implantable device, and generating an alarm message on display 159. Controller 152 also may include firmware for transmitting commands input using input device 164 to the implantable device, and monitoring operation of the implantable device during execution of such commands, for example, during boost or jogging/shaking operation of the gear pump to clear a blockage. In addition, controller 152 controls and monitors various power operations of handpiece 151, including operation of inductive circuit 156 during recharging of the implantable device, displaying the state of charge of battery 74, and controlling charging and display of state of charge information for battery 157.

Nonvolatile memory 153 preferably comprises flash memory or EEPROM, and stores the unique device identifier for its associated implantable device, firmware to be executed by controller 152, configuration set point, and optionally, coding to be executed on transceiver 155 and/or inductive circuit 156. Firmware and set point data stored on nonvolatile memory 153 may be updated using information supplied by control and monitoring system 40 via port 162. Volatile memory 154 is coupled to and supports operation of controller 152, and stores data and event log information uploaded from implantable device 20.

In addition, in a preferred embodiment, nonvolatile memory 153 stores programming that enables the charging and communication system to perform some initial start-up functions without communicating with the monitor and control system. In particular, memory 153 may include routines that make it possible to test the implantable device during implantation using the charging and communication system alone in a “self-prime mode” of operation. In this case, a button may be provided that allows the physician to manually start the pump, and display 159 is used to provide feedback whether the pumping session was successful or not. Display 159 of the charging and communication system also may be used to display error messages designed to assist the physician in adjusting the position of the implantable device or peritoneal or bladder catheters. These functions preferably are disabled after the initial implantation of the implantable device.

Transceiver 155 preferably comprises a radio frequency transceiver, e.g., conforming to the Bluetooth or IEEE 802.11 wireless standards, and is configured for bi-directional communications via antenna 160 with transceiver circuit 76 disposed in the implantable device. Transceiver 155 also may include a low power mode of operation, such that it periodically awakens to listen for incoming messages and responds only to those messages including the unique device identifier assigned to its associated implantable device. Transceiver 155 preferably employs an encryption routine to ensure that messages sent to, or received from, the implantable device cannot be intercepted or forged.

Inductive circuit 156 is coupled to coil 163, and is configured to inductively couple with coil 84 of the implantable device to recharge battery 74 of the implantable device. In one embodiment, inductive circuit 156 is coupled to indicator 158, preferably a plurality of LEDs that light to indicate the extent of magnetic coupling between coils 163 and 84 (and thus quality of charging), thereby assisting in positioning handpiece 151 relative to the implantable device. In one preferred embodiment, inductive coils 84 and 163 are capable of establishing good coupling through a gap of 35 mm, when operating at a frequency of 315 kHz or less. In an embodiment in which implantable device includes optional infrared LED 83, charging and communication system 30 may include an optional infrared sensor (not shown) which detects that infrared light emitted by LED 83 and further assists in positioning handpiece 151 to optimize magnetic coupling between coils 163 and 84, thereby improving the energy transmission to the implantable device.

Controller 152 also may be configured to periodically communicate with the implantable device to retrieve temperature data generated by temperature sensor 78 and stored in memory 72 during inductive charging of battery 74. Controller 152 may include firmware to analyze the battery temperature, and to adjust the charging power supplied to inductive circuit 163 to maintain the temperature of the implantable device below a predetermined threshold, e.g., less than 2 degrees C. above body temperature. That threshold may be set to reduce thermal expansion of the battery and surrounding electronic and mechanical components, for example, to reduce thermal expansion of motor and gear pump components and to reduce the thermal strain applied to the seal between lower portion 92 of housing and upper housing 93. In a preferred embodiment, power supplied to inductive coil 163 is cycled between high power (e.g., 120 mA) and low power (e.g., 40 mA) charging intervals responsive to the measured temperature within the implantable device.

As discussed above with respect to inductive circuit 75 of the implantable device, inductive circuit 156 optionally may be configured to transfer additional power to motor 73 of the implantable device, via inductive circuit 75 and battery 74, in a “boost” mode or jogging mode to unblock the gear pump. In particular, if an alarm is transmitted to smartphone 50 or controller 152 that motor 73 is stalled, e.g., due to a block created by viscous fluid, the patient may be given the option of using smartphone 50 or input device 164 to apply an overvoltage to motor 73 from battery 74 and/or inductive circuit 75 for a predetermined time period to free the blockage. Alternatively, activating input device 164 may cause controller 152 to command processor 70 to execute a routine to jog or shake the gear pump by rapidly operating motor 74 in reverse and forward directions to disrupt the blockage. Because such modes of operation may employ higher energy consumption than expected during normal operation, inductive circuits 156 and 75 may be configured to supply the additional energy for such motor operation directly from the energy stored in battery 157, instead of depleting battery 74 of the implantable device.

Battery 157 preferably comprises a lithium ion or lithium polymer battery capable of long lasting operation, e.g., up to three years. Battery 157 has sufficient capacity to supply power to handpiece 151 to operate controller 152, transceiver 155, inductive circuit 156 and the associated electronics while disconnected from base 31 and during charging of the implantable device. In a preferred embodiment, battery 157 has sufficient capacity to fully recharge battery 74 of the implantable device from a depleted state in a period of about 2-4 hours. Battery 157 also should be capable of recharging within about 2-4 hours. It is expected that for daily operation moving 700 ml of fluid, battery 157 and inductive circuit 156 should be able to transfer sufficient charge to battery 74 via inductive circuit 75 to recharge the battery within about 30 minutes. Battery capacity preferably is supervised by controller 152 using a charge accumulator algorithm.

Referring again to FIGS. 7A and 7B, handpiece 151 preferably includes housing 165 having multi-function button 166 (corresponding to input device 164 of FIG. 8) and display 167 (corresponding to display 159 of FIG. 8). A plurality of LEDs 168 is disposed beneath a translucent portion of handpiece 151, and corresponds to indicator 158 of FIG. 8. Port 169 enables the handpiece to be coupled to monitoring and control system 40 (and corresponds to port 162 of FIG. 8), while connector 170 (corresponding to connector 161 in FIG. 8) permits the handpiece to be coupled to base 31 to recharge battery 157. Multi-function button 166 provides the patient the ability to input a limited number of commands to the implantable device. Display 167, preferably an OLED or LCD display, provides visible confirmation that a desired command input using multifunction button 166 has been received. Display 167 also may display the status and state of charge of battery 74 of the implantable device, the status and state of charge of battery 157 of handpiece 151, signal strength of wireless communications, quality-of-charging, error and maintenance messages. Inductive coil portion 171 of housing 165 houses inductive coil 163.

LEDs 168 are visible through the material of housing 165 when lit, and preferably are arranged in three rows of two LEDs each. During charging, the LEDs light up to display the degree of magnetic coupling between inductive coils 163 and 84, e.g., as determined by energy loss from inductive circuit 156, and may be used by the patient to accurately position handpiece 151 relative to the implantable device. Thus, for example, a low degree of coupling may correspond to lighting of only two LEDs, an intermediate degree of coupling with lighting of four LEDs, and a preferred degree of coupling being reflected by lighting of all six LEDs. Using this information, the patient may adjust the position of handpiece 151 over the area where implantable device is located to obtain a preferred position for the handpiece, resulting in the shortest recharging time. In one preferred embodiment, LEDs 168 are replaced with an analog bar display on display 167, which indicates the quality of charge coupling.

### Monitoring and Control System

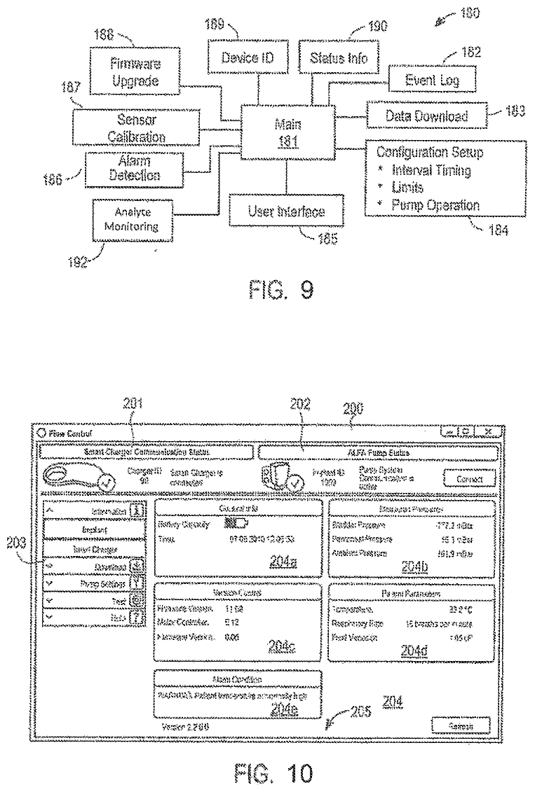

Turning to FIG. 9, the software implementing monitoring and control system 40 of FIG. 1 will now be described. Software 180 comprises a number of functional blocks, schematically depicted in FIG. 9, including main block 184, event logging block 182, data download block 183, configuration setup block 184, user interface block 185, alarm detection block 186 including health monitor block 191 and analyte monitoring block 192, sensor calibration block 187, firmware upgrade block 188, device identifier block 189 and status information block 190. In one embodiment, the software is coded in C++ and employs an object oriented format, although other software languages and environments could be used. In one embodiment, the software is configured to run on top of a Microsoft Windows® (a registered trademark of Microsoft Corporation, Redmond, Wash.) or Unix-based operating system, such as are conventionally employed on desktop and laptop computers, although other operating systems could be employed.

The computer running monitoring and control system software 180 preferably includes a data port, e.g., USB port or comparable wireless connection that permits handpiece 151 of the charging and communication system to be coupled via port 169. Alternatively, as discussed above, the computer may include a wireless card, e.g., conforming to the IEEE 802.11 standard, thereby enabling handpiece 151 to communicate wirelessly with the computer running software 180. As a further alternative, the charging and communication system may include telephony circuitry that automatically dials and uploads data, such as alarm data, from handpiece 151 to a secure website accessible by the patient's physician.

Main block 184 preferably consists of a main software routine that executes on the physician's computer, tablet or smartphone, and controls overall operation of the other functional blocks. Main block 184 enables the physician to download event data and alarm information stored on handpiece 151 to their computer, tablet or smartphone, and also permits control and monitoring software 180 to directly control operation of the implantable device when coupled to handpiece 151. Main block also enables the physician to upload firmware updates and configuration data to the implantable device.

Event Log block 182 is a record of operational data downloaded from the implantable device via the charging and communication system, and may include, for example, pump start and stop times, motor position, sensor data for the peritoneal cavity and bladder pressures, patient temperature, respiratory rate or fluid temperature, pump outlet pressure, humidity, pump temperature, battery current, battery voltage, battery status, and the like. The event log also may include the occurrence of events, such as pump blockage, operation in boost or jog modes, alarms or other abnormal conditions.

Data Download block 183 is a routine that handles communication with handpiece 151 to download data from volatile memory 154 after the handpiece is coupled to the computer running monitoring and control software 180. Data Download block 183 may initiate, either automatically or at the instigation of the physician via user interface block 185, downloading of data stored in the event log.

Configuration Setup block 184 is a routine that configures the parameters stored within nonvolatile memory 71 that control operation of the implantable device. The interval timing parameters may determine, e.g., how long the processor remains in sleep mode prior to being awakened to listen for radio communications or to control pump operation. The interval timing parameters may control, for example, the duration of pump operation to move fluid from the peritoneal cavity to the bladder, the pump speeds to be used during specific phases of the DSR therapy, and the interval between periodic tick movements that inhibit blockage of the implantable device and peritoneal and bladder catheters. Interval timing settings transmitted to the implantable device from monitoring and control software 180 also may determine when and how often event data is written to nonvolatile memory 71, and to configure timing parameters used by the firmware executed by processor 152 of handpiece 151 of the charging and communication system. Block 184 also may be used by the physician to configure parameters stored within nonvolatile memory 71 relating to limit values on operation of processor 70 and motor 73, and to set target and threshold values. These values may include the sodium concentration detected in the peritoneal catheter at which fluid transfer to the bladder should be initiated, maximum DSR solution dwell time, minimum and maximum pressures at the peritoneal and bladder catheters, the maximum temperature differential during charging, times when the pump may and may not operate, etc. The limit values set by block 184 also configure parameters that control operation of processor 152 of handpiece 151.

Block 184 also may configure parameters store within nonvolatile memory 71 of the implantable device relating to control of operation of processor 70 and motor 73. These values may include target volumes of fluid to transport, volume of fluid to be transported per pumping session, motor speed and duration per pumping session. Block 184 also may specify the parameters of operation of motor 73 during boost mode of operation and shake/jog modes of operation. Such parameters may include motor speed and voltage, duration/number of revolutions of the motor shaft when alternating between forward and reverse directions, etc.

User interface block 185 handles display of information retrieved from the monitoring and control system and implantable device via data download block 183, and presents that information in an intuitive, easily understood format for physician review. As described below with respect to FIG. 10, such information may include status of the implantable device, status of the charging and control system, measured pressures, volume of fluid transported per pumping session or per day, etc. User interface block 185 also generates user interface screens that permit the physician to input information to configure the interval timing, limit and pump operation parameters discussed above with respect to block 184.

Alarm detection block 186 may include a routine for evaluating the data retrieved from the implantable device or charging and communication system, and flagging abnormal conditions for the physician's attention, and determining whether a specific alarm condition should be reported to smartphone 50 to prompt immediate patient action. Alarm detection block 186 also may include health monitor block 191, which is configured to alert the patient or physician to any changes in the patient's health that may warrant changing the volume, time, and/or frequency with which the DSR therapy is conducted.

Sensor calibration block 187 may include routines for testing or measuring drift, of sensors 70, 78-81 and 85 employed in the implantable device, e.g., due to aging or change in humidity. Block 187 may then compute offset values for correcting measured data from the sensors, and transmit that information to the implantable device for storage in nonvolatile memory 71. For example, pressure sensors 104a-104d may experience drift due to aging or temperature changes. Block 187 accordingly may compute offset values that are then transmitted and stored in the implantable device to account for such drift.

Firmware upgrade block 188 may comprise a routine for checking the version numbers of the processor or motor controller firmware installed on the implantable device and/or processor firmware on charging and communication system, and identify whether upgraded firmware exists. If so, the routine may notify the physician and permit the physician to download revised firmware to the implantable device for storage in nonvolatile memory 71 or to download revised firmware to the charging and communication system for storage in nonvolatile memory 153.

Device identifier block 189 consists of a unique identifier for the implantable device that is stored in nonvolatile memory 71 and a routine for reading that data when the monitoring and control system is coupled to the implantable device via the charging and communication system. As described above, the device identifier is used by the implantable device to confirm that wireless communications received from a charging and communication system are intended for that specific implantable device. Likewise, this information is employed by handpiece 151 of the charging and communication system in determining whether a received message was generated by the implantable device associated with that handpiece. Finally, the device identifier information is employed by monitoring and control software 180 to confirm that the handpiece and implantable device constitute a matched set.

Status information block 190 comprises a routine for interrogating implantable device, when connected via handpiece 151, to retrieve current status date from the implantable device, and/or handpiece 151. Such information may include, for example, battery status, the date and time on the internal clocks of the implantable device and handpiece, version control information for the firmware and hardware currently in use, and sensor data. Status information block 190 also may make some or all of the preceding information available to smartphone 50 for display to the patient.

Referring now to FIG. 10, an exemplary screen shot generated by user interface block 185 of software 180 is described for an implantable system used in accordance with the methods of the present invention for conducting DSR therapy. FIG. 10 shows main screen 200, which may be displayed on monitoring and control system 40, and includes a status area that displays status information retrieved from the implantable device and the charging and communication system by the routine corresponding to block 190 of FIG. 9. More particularly, the status area includes status area 201 for the charging and communication system (referred to as the “Smart Charger) and status area 202 for the implantable device (referred to as the “ALFA Pump”). Each status area includes an icon showing whether the respective system is operating properly, indicated by a checkmark, the device identifier for that system, and whether the system is connected or active. If a parameter is evaluated by the alarm detection block 186 to be out of specification, the icon may instead include a warning symbol. Menu bar 203 identifies the various screens that the physician can move between by highlighting the respective menu item. Workspace area 204 is provided below the status area, and includes a display that changes depending upon the menu item selected. Below workspace area 204, navigation panel 205 is displayed, which includes the version number of software 180 and a radio button that enables the displays in workspace area 204 to be refreshed.

In FIG. 10, the menu item “Information” with submenu item “Implant” is highlighted in menu bar 203. For this menu item selection, workspace area 204 illustratively shows, for the implantable device, battery status window 204a, measured pressures window 204b and firmware version control window 204c. Battery status window 204a includes an icon representing the charge remaining in battery 74, and may be depicted as full, three-quarters, one-half, one-quarter full or show an alarm that the battery is nearly depleted. The time component of window 204a indicates the current time as received from the implantable device, where the date is expressed in DD/MM/YYYY format and time is expressed in HR/MIN/SEC format based on a 24-hour clock. Measured pressures window 204b displays the bladder pressure, peritoneal pressure and ambient pressures in mBar measured by sensors 104a, 104b and 104d respectively (see FIG. 4A). Version control window 204c indicates the firmware version for processor 70, for the motor controller, and the hardware version of the implantable device. Patient parameters window 204d displays the patient's temperature, respiratory rate, and intra-abdominal pressure. Note that if implantable device included other types of sensors, e.g., sensors that measure the levels of fluid in the body, then the parameters measured by such sensors could also be displayed in window 204d.

Alarm condition window 204e displays any changes in parameters that may indicate a change in the patient's health, such as the possible development of heart failure decompensation or an improvement or worsening of the patient's health (Block 186 in FIG. 9). For example, as illustrated, alarm condition window 204e may alert the patient and/or physician that the patient's intra-abdominal pressure is abnormally high, so that the patient and physician then may follow up to rectify that situation. In some embodiments, based on information displayed in windows 204b, 204d, and/or 204e, the physician may adjust the operating parameters of the pump.

### Arrangements for Handling DSR Solution

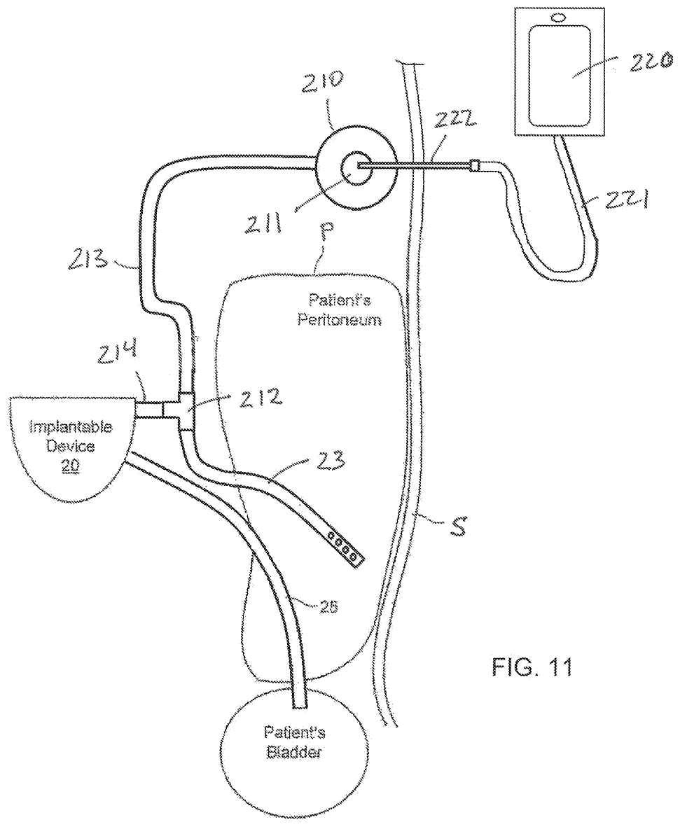

Referring now to FIG. 11, a first catheter arrangement for instilling fluid into a patient's peritoneal cavity at the initiation of a DSR therapy session is described. Device 20 is implanted subcutaneously, preferably outside of the patient's peritoneal cavity as defined by peritoneal membrane P. Implantable device 20 is placed beneath skin S so that the device may readily be charged by, and communicate with, charging and communication system 30. Subcutaneous port 210 includes a self-healing membrane 211 and is implanted in the patient's upper abdomen, and is coupled to peritoneal catheter 23 by tee connector 212 and instillation line 213. Tee connector 212 is coupled to the inlet port of implantable device 20 by tubing 214. The outlet port of implantable device 20 is coupled to bladder catheter 25, thereby enabling implantable device 20 to transfer fluid from the peritoneal cavity to the patient's bladder at the completion of the DSR therapy session.

In accordance with one aspect of the invention, tee connector 212 optionally may include a valve, that selectively permits fluid to flow from subcutaneous port 210, through instillation line 213 and into peritoneal cavity P via peritoneal catheter 23, but prevents instilled DSR solution from passing to the inlet port of implantable device 20. Sterile DSR solution disposed in bag 220, is coupled via tubing 221 to non-coring needle 222, which may be inserted through patient's skin S and self-healing membrane 211 to place the contents of bag 220 in fluid communication with the interior of subcutaneous port 210. In this manner, DSR solution from bag 220, typically 0.5 up to 2 liters, will flow through tubing 221 and needle 222 into subcutaneous port 210, and then into the peritoneal cavity via instillation line 213, tee junction 212 and peritoneal catheter 23. The DSR solution thus instilled with initiate the DSR therapy session while simultaneously flushing peritoneal catheter 23 of debris that could potentially block the catheter when the DSR and ultrafiltrate subsequently is withdrawn.

After instillation of the DSR solution from bag 220 is completed, needle 221 is withdrawn. Later, upon completion of the dwell period, for example, as may be determined by expiration of a specified time interval, detected analyte concentration, or rate of change of intraabdominal pressure, implantable device 20 is actuated to suck fluid from the peritoneal cavity via the peritoneal catheter, tee junction 212 and tubing 214 to implantable device 20. No fluid is drawn through instillation line 213 as the valve in tee-connector 212 isolates the instillation line from pump suction. Fluid drawn into peritoneal catheter 23 is pumped by implantable device 20 through bladder catheter 25 and into the patient's bladder.

Alternatively, tee connector 212 may be employed without a valve. In this case, some DSR solution may be lost as directly flowing into the bladder through implantable device 20, and not into the peritoneum. However, it is expected that the gears of the implantable device 20 will impede fluid loss through the pump, and accordingly the loss would be small. Moreover, the expected DSR loss through the pump during instillation could be empirically determined, and thus taken into consideration for dosing of the DSR solution for a given DSR therapy session. For example, if was empirically known that 100 ml is lost during infusion of 1000 ml into the abdomen, an 1100 ml total dose may be proscribed for infusion. On the other hand, during operation of implantable device 20 to move fluid from the peritoneal cavity to the bladder, suction induced by the pump would not pose a problem for instillation line 213 or subcutaneous port 210 because the subcutaneous port will remain closed via operation of self-healing membrane 211.

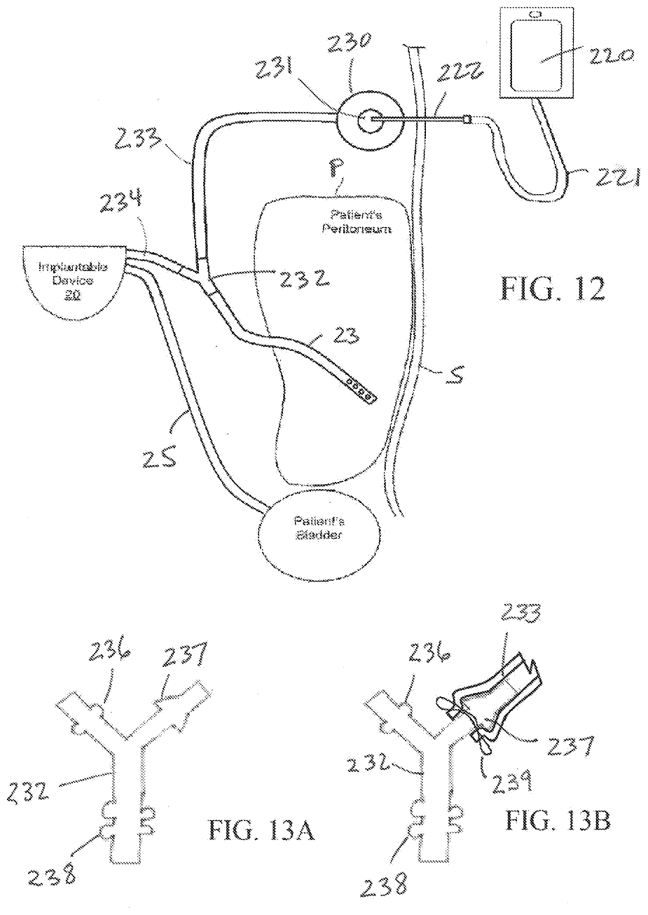

Referring now to FIGS. 12, 13A and 13B, an alternative arrangement for instilling DSR fluid into a patient's peritoneum is described. As for the arrangement of FIG. 11, device 20 is implanted subcutaneously, preferably outside of the patient's peritoneal cavity as defined by peritoneal membrane P. Implantable device 20 is placed beneath skin S so that the device may readily be charged by, and communicate with, charging and communication system 30. Subcutaneous port 230 includes a self-healing membrane 231 and is implanted in the patient's upper abdomen, and is coupled to peritoneal catheter 23 by Y-connector 232 and instillation line 233. Y-connector 232 is coupled to the inlet port of implantable device 20 by tubing 234. The outlet port of implantable device 20 is coupled to bladder catheter 25, thereby enabling implantable device 20 to transfer fluid from the peritoneal cavity to the patient's bladder at the completion of the DSR therapy session.

Y-connector 212 is configured to permit fluid to flow from subcutaneous port 230, through instillation line 231 and into peritoneal cavity P via peritoneal catheter 23, but reduces the amount of instilled DSR solution that can pass to the inlet port of implantable device 20. As for the preceding arrangement, sterile DSR solution disposed in bag 220, is coupled via tubing 221 to non-coring needle 222, which may be inserted through patient's skin S and self-healing membrane 231 to place the contents of bag 220 in fluid communication with the interior of subcutaneous port 230. Accordingly, DSR solution from bag 220, typically 0.5 up to 2 liters, will flow through tubing 221 and needle 222 into subcutaneous port 230, and then into the peritoneal cavity via instillation line 233, Y-connector 232 and peritoneal catheter 23. DSR solution instilled in this matter at the start of the DSR therapy session simultaneously will flush peritoneal catheter 23 of debris that could potentially block the catheter when the DSR and ultrafiltrate subsequently is withdrawn.

After instillation of the DSR solution from bag 220 is completed, needle 221 is withdrawn. Later, upon completion of the dwell period, as described above, implantable device 20 is actuated to suck fluid from the peritoneal cavity via peritoneal catheter 23, Y-connector 232 and tubing 234 to implantable device 20. The configuration of Y-connector 232 preferably is designed so that during delivery of DSR solution through instillation line 233, hydraulic forces induced by the flow through the Y-connector will reduce lost flow to the bladder through implantable device 20. In particular, when pressure is applied to instillation line 233 coupled to the Y-connector, based on fluid dynamics principles, the vast majority of flow and pressure will exit the lower end of Y-connector 232 towards peritoneal catheter 23, which is thus flushed. Relatively little is expected to reach “backwards” into the other upper end of the Y-connector and thus slip-flow through the pump is reduced. Moreover, as for the embodiment of the tee connector without a valve, it is expected that the gears of the implantable device 20 will impede fluid loss through the pump, thus further reducing loss of DSR loss through the pump during instillation. In addition, such lost amounts could be empirically determined, such that a slightly larger dose of DSR solution may be proscribed for infusion. Fluid drawn into peritoneal catheter 23 is pumped by implantable device 20 through bladder catheter 25 and into the patient's bladder.

Referring now to FIGS. 13A and 13B, another aspect of the Y-connector is described. As depicted in FIG. 13A, the Y-connector may be supplied as a separate component of the catheter arrangement, which is assembled by the implanting surgeon or physician when implantable device 20 is first implanted in the patient. As depicted in FIG. 13A, each end of Y-connector 232 should have circumference protuberances 236, 237 and 238 which serve to retain line 233, tubing 234 and catheter 23 installed onto the respective ends of the Y-connector. As shown in FIG. 13B, the physician or surgeon also may apply suture 239 through line 233, tubing 234 and catheter 23 during the implantation procedure to tightly close and bind those components to Y-connector 232, thus ensuring that the components are not pulled free from the Y-connector during normal expected use. Alternatively, a plastic zip tie or similar device may be used in lieu of sutures to join the various tubing components to the Y-connector. As a still further alternative, the connector may include snap rings or other suitable structure to fasten the tubes to the Y-connector to secure the tubing from separating.

In accordance with a further aspect of the invention, the implantable device could be programmed to include a flush feature, which could be implemented by a command issued from smartphone 50, charging and communication system 30 or monitoring and control system 40 to avoid exposing implantable device 20 to excessive high pressures during instillation for the DSR solution and flushing of peritoneal catheter 23. In particular, issuing the command to implantable device 20 could activate the built-in pressure sensors monitor the applied pressure and report to a connected charging and control system 30 or smartphone 50. If detected pressure is zero or low, a signal indicating acceptable status could be sent to the connected device. However, once a threshold pressure is attained that would be dangerous to the pump mechanics, or the patient, a warning signal could be provided to the connected device. If pressure continued to rise further, an alarm condition could be generated, indicating to the patient that the flushing pressure needs to be immediately reduced.

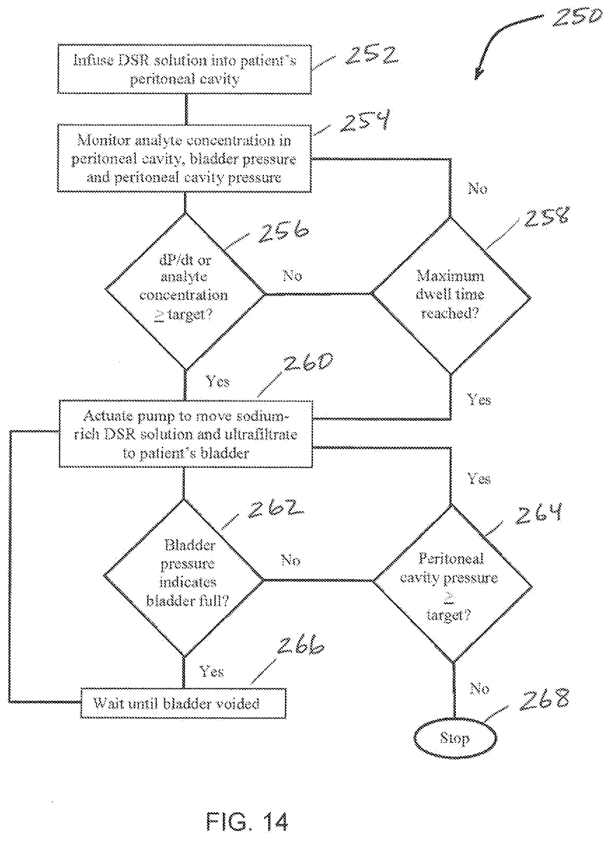

Referring now to FIG. 14, methods of using the implantable system of FIG. 1 to conduct a DSR therapy session are described. Method 250 includes introducing no or low sodium DSR solution into the peritoneal cavity, for example, using an arrangement as described above with respect to FIG. 11 or 12. A sufficient amount of DSR solution, generally 0.5 up to about 2 liters, is introduced into the peritoneal cavity of the patient and allowed to a desired threshold is attained, such as expiration of a specified dwell time, detection of a target analyte concentration, or intraabdominal pressure. As described in the above-incorporated patent and application, the goal of the DSR therapy session is to draw excess fluid and/or sodium from the patient's body tissues into the peritoneal cavity, from which it is removed to the bladder via implantable device 20.

In step 254, a number of physiologic parameters may be monitored as indicative of the status of the DSR therapy session, including the analyte concentration in the patient's peritoneal cavity, the intraabdominal pressure and bladder pressure. At step 256, the rate of change with time of the measured analyte concentration in the DSR solution and fluid accumulated in the peritoneal cavity may be compared to a target value. For example, if the rate of change of the sodium concentration within the fluid in the peritoneal cavity is initially high but begins to decrease over time, that observed value may indicate further extending the dwell time will not result in significant additional fluid or sodium migration to the peritoneum, and thus the current session should be ended. That determination then may be used to initiate pumping of fluid from the peritoneal cavity to the patient's bladder.

The target value may be downloaded to implantable device by the physician using software monitoring and control system 40 via external charging and communication system 30 prior to initiating the DSR therapy session. Alternatively, the target value may be static, i.e., programmed one time, or may be revised prior to each DSR therapy session based on prior results for that patient and titrated to provide a targeted blood sodium serum level by completion of the DSR therapy session. Alternatively, the target value may consist of another value of interest for a particular patient, such as a target quantity of sodium to be removed during the DSR therapy session, or a target value of an infusate concentration at which pumping is to begin and/or cease.

If at step 256 the monitored value is less than the target value (“No” branch at step 256), the processor of implantable device 20 then may evaluate at step 258 whether the current dwell time for the DSR solution exceeds the maximum dwell time for that DSR therapy session. The maximum dwell time may be a static value that is programmed once, or a value that is set prior to each DSR therapy session as may be determined by the physician. If the current dwell time is less than the maximum value (“No” branch at step 258), the processor may internally set another time interval, e.g., 5 or 10 minutes, after which it will continue to monitor the specified parameter at step 254 and evaluate whether the target has been attained at step 256. If however, the maximum dwell time has been exceeded at step 258, then the processor will activate the pump of implantable device 20 to begin transferring sodium-rich DSR solution and ultrafiltrate from the peritoneal cavity to the patient's bladder, at step 260 (“Yes” branch at step 258). Referring back to the decision box at step 256, if the monitored value equals or exceeds the target value (the “Yes” branch at step 256), the processor also will activate the pump of implantable device 20 at step 260.

Once the pump of implantable device 20 is activated, it will transfer fluid from the peritoneal cavity to the bladder via bladder catheter 25. To ensure that the pump motor does not overheat from continuous operation, the pump may run for a predetermined specified interval, e.g., 5 or 10 minutes, then rest for a brief interval, e.g., 2-5 minutes, and then resume operation. Alternatively, the pump may run at different speeds for different intervals of the pumping period. If the pump is actuated periodically in this manner to complete removal of the DSR solution and accumulated ultrafiltrate from the peritoneal cavity, for example, as determined by the pressure in the peritoneal cavity falling below a target value, the processor may no longer require that the detected physiologic parameter(s) exceed the target level for that particular DSR therapy session. Accordingly, once the threshold is satisfied or the dwell time exceeded for a particular DSR therapy session, the pump will be actuated intermittently until drainage of the peritoneal cavity is determined to be complete.

Apart from intermittent operation to permit brief periods for the pump to rest, or operation at different flow rates, as described above, the processor also monitors the patient's bladder pressure to ensure that transfer of fluid to the bladder is interrupted when the bladder is deemed to be full. Fullness may be determined by the physician receiving oral feedback of discomfort from the patient during initial set-up and programming of implantable device 20. Accordingly, at step 262, bladder pressure is monitored, particularly at times when fluid is being transferred to the bladder. If the bladder pressure is lower than the level associated with bladder fullness, as initially programmed (“No” branch at step 262), the processor next evaluates whether the pressure in the peritoneal cavity is greater than a predetermined target pressure at step 264. The target pressure at step 264 is a pressure determined to correspond to fluid accumulation in the patient's peritoneal cavity, which value may set during initial implantation and then periodically updated by the physician using monitoring and control system 40 during the course of multiple DSR therapy sessions. If at step 264 that peritoneal pressure indicates that peritoneal cavity still contains fluid to be transferred to the bladder, the pump of implantable device 20 may remain active, change speeds, or if at rest, resume operation to transfer fluid to the bladder (“Yes” branch at step 264). If the peritoneal cavity pressure is determined at step 264 to be less than target pressure (“No” branch at step 264), the processor will deactivate the pump and store a record reflecting completion of that DSR therapy session in the non-volatile memory for eventual transmission to event log 182 (see FIG. 9).

Referring again to step 262, if the processor of implantable device 20 detects that the pressure of fluid in the patient's bladder exceeds the target value (“Yes” branch at step 262), the processor will cease pumping and movement of fluid to the patient's bladder. The processor then will set a timer, at step 266, during which implantable device 20 waits until the patient voids his bladder. Once the patient voids his bladder and the bladder pressure drops below the target level, the processor again activates the pump to transfer remaining fluid in the peritoneal cavity to the bladder.

As described above, actuation of the pump transfers sodium-laden DSR solution and ultrafiltrate from the peritoneal cavity to the bladder, thereby reducing the level of sodium in the body and causing elimination of excess fluid by i) enhancing normal kidney function through urination and ii) removal to the bladder of osmotic ultrafiltrate that accumulates in the peritoneal cavity. It is expected that by configuring to activate the pump responsive to a monitored parameter of the fluid in the peritoneal cavity, better control of serum sodium concentration may be maintained than could be achieved by basing the action of the DSR solution on dwell time alone.

Energy may be wirelessly transferred to the implantable device, and data received from the device, using charging and communication system 30 described above with reference to FIG. 1. For example, the implantable device may record parameters reflective of the health of the patient and the operation of the device, which parameters may be communicated to the charging and communication system or patient's smartphone. The data, e.g., parameters recorded by the implantable device, also may be provided to monitoring and control software 40, via the charging and communication system. Based on those parameters, the health of the patient may be assessed using the software, and the physician may remotely communicate any modifications to the target analyte concentration, target pressures, flow rates, volume, dwell time, or frequency with which the implantable device is activated to transfer DSR solution and ultrafiltrate containing the extracted sodium to the bladder. Such communication may be performed via the charging and communication system.

While various illustrative embodiments of the invention are described above, it will be apparent to one skilled in the art that various changes and modifications may be made therein without departing from the invention. For example, the implantable device may be ordered differently and may include additional or fewer components of various sizes and composition. The appended claims are intended to cover all such changes and modifications that fall within the true spirit and scope of the invention.

What is claimed is:

1. A system for use with a DSR solution for conducting direct sodium removal therapy in a patient, the system comprising:
an implantable device including a pump, a transceiver, a battery and a processor operably coupled to the pump, transceiver and battery, the pump having an inlet port and an outlet port;
a peritoneal catheter connected having a first end configured to be disposed in a peritoneal cavity of the patient and a second end configured to be coupled in fluid communication to the inlet port of the pump;
a bladder catheter having an inlet end configured to be coupled to the outlet port of the pump and an outlet end configured to be disposed in a urinary bladder of the patient,
a sensor configured to output a signal indicative of a monitored parameter of an infusate instilled into a peritoneal cavity of the patient, the sensor operably coupled to the processor,
wherein the processor is configured to execute programmed instructions to:
monitor the output of the sensor, and
control actuation of the pump to move fluid from the peritoneal cavity to the urinary bladder responsive either to the output of the analyte sensor or after expiration of a predetermined dwell time,
wherein the processor is programmed to selectively vary the speed of the pump during actuation.

2. The system of claim 1, wherein the sensor is configured to a rate of change of an analyte concentration within the peritoneal cavity.

3. The system of claim 1, wherein the implantable device further comprises one or more sensors for monitoring operational status of the pump, and wherein the processor further is programmed to generate an alarm condition when the operational status of the pump indicates a fault.

4. The system of claim 3, further comprising a patient smartphone having an application configured to communicate with the transceiver of the implantable device to report the alarm condition.

5. The system of claim 4, wherein the application further is programmed to enable the patient to issue commands to the implantable device via the transceiver.

6. The system of claim 1, further comprising an instillation line configured to be coupled at a first end to a subcutaneous port having a self-healing membrane and at a second end to a first side of a tee connector, wherein a second side of the tee connector is coupled to the second end of the peritoneal cavity and a third side of the tee connector is coupled to the inlet port of the pump.

7. The system of claim 6, wherein the tee connector further comprises a valve.

8. The system of claim 1, further comprising an instillation line configured to be coupled at a first end to a subcutaneous port having a self-healing membrane and at a second end to a first side of a Y-connector, wherein a second side of the Y-connector is coupled to the second end of the peritoneal cavity and a third side of the tee connector is coupled to the inlet port of the pump.

9. The system of claim 1, further comprising an external charging and communications system configured to inductively charge the battery, wherein the external charging and communications system is configured to periodically couple to a monitoring and control system to exchange data generated by the implantable device.

10. The system of claim 1, wherein the pump is a motor-driven gear pump.

11. A method of performing DSR therapy to remove excess sodium from a patient to reduce fluid overload, the method comprising,
implanting in a patient an implantable device including a pump, a sensor and a processor operably coupled to the pump and the sensor, the pump having an inlet port and an outlet port;
implanting in the patient a peritoneal catheter with a first end disposed in a peritoneal cavity of the patient and a second end in fluid communication with the inlet port of the pump;
implanting a bladder catheter with an inlet end coupled to the outlet port of the pump and an outlet end disposed in a urinary bladder of the patient,
infusing a DSR solution having no or low sodium into the peritoneal cavity;
monitoring with the sensor a constituent of the DSR solution and accumulated ultrafiltrate in the peritoneal cavity; and
controlling operation of the pump to move the DSR solution and the accumulated ultrafiltrate from the peritoneal cavity to the urinary bladder responsive to the output of the analyte sensor, wherein the speed of the pump is selectively varied during operation.

12. The method of claim 11, further comprising triggering the pump to move fluid from the peritoneal cavity to the urinary bladder responsive a detected rate of change of an analyte concentration within the peritoneal cavity.

13. The method of claim 11, wherein the implantable device further comprises one or more sensors for monitoring operational status of the pump, the method further comprising generating an alarm condition when the operational status of the pump indicates a fault.

14. The method of claim 13, further comprising providing a patient smartphone having an application configured to communicate with a transceiver of the implantable device, the method further comprising reporting the alarm condition on a display of the smartphone.

15. The method of claim 14, wherein the application is programmed to enable the patient to issue commands to the implantable device via the transceiver, the method further comprising issuing a command to resolve the fault.

16. The method of claim 11, further implanting an instillation line coupled at a first end to a subcutaneous port having a self-healing membrane and at a second end to a first side of a tee connector, implanting a second side of the tee connector coupled to the second end of the peritoneal cavity and implanting a third side of the tee connector coupled to the inlet port of the pump.

17. The method of claim 16, wherein implanting the tee connector comprises implanting a tee connector having a valve.

18. The method of claim 16, further implanting an instillation line coupled at a first end to a subcutaneous port having a self-healing membrane and at a second end to a first side of a Y-connector, implanting a second side of the Y-connector coupled to the second end of the peritoneal cavity and implanting a third side of the Y-connector coupled to the inlet port of the pump.

19. The method of claim 11, further comprising inductively charging a battery of the implantable device using an external charging and communications system, and periodically coupling the external charging and communications system to a monitoring and control system to exchange data generated by the implantable device

20. The method of claim 11, wherein the pump of the implantable device comprises a motor-driven gear pump, the method further comprising varying a voltage applied to the motor-driven gear pump to vary an output flow rate of the pump.
